(19) United States
(12) Patent Application Publication
(10) Pub. No.: US 2008/0260753 A1
(43) Pub. Date: Oct. 23, 2008

(54) Mouse Models of Crohn's Disease and a Method to Develop Specific Therapeutics

(75) Inventors: Michael Karin, La Jolla, CA (US); Shin Maeda, Tokyo (JP); Laurie A. Bankston, Solana Beach, CA (US)

Correspondence Address: BUCHANAN, INGERSOLL & ROONEY LLP, P.O. BOX 1404, ALEXANDRIA, VA, 22313-1404, US

(73) Assignee: The Regents of the University of California, Oakland, CA (US)

(21) Appl. No.: 11/547,821

(22) PCT Filed: Apr. 8, 2005
(86) PCT No.: PCT/US2005/011798

§ 371 Date: Jul. 7, 2008

Related U.S. Application Data
(60) Provisional application No. 60/560,916, filed on Apr. 9, 2004.

(51) Int. Cl. A61K 39/395; A61K 38/05; A61K 31/721; C12N 15/85; A61P 1/00; A01K 67/027; C12N 5/06; G01N 33/00

(52) U.S. Cl. 424/172.1; 514/19; 514/59; 800/21; 800/9; 435/325; 800/3

(57) ABSTRACT

Provided are compositions, transgenic animals and methods for screening and analyzing agents useful for treating inflammatory bowel diseases. Also provided are methods to treat inflammatory bowel disease, Crohn's disease and Blau syndrome.

### CROSS REFERENCE TO RELATED APPLICATIONS

This application claims priority under 35 U.S.C. §119 from Provisional Application Ser. No. 60/560,916, filed Apr. 9, 2004, the disclosure of which is incorporated herein by reference.

### STATEMENT REGARDING FEDERALLY SPONSORED RESEARCH

The invention was funded in part by Grant Nos. AI043477 and DK035108 awarded by the National Institutes of Health (NIH). The government may have certain rights in the invention.

### TECHNICAL FIELD

This invention relates to transgenic organisms, more particularly related to knockout and/or mutant organisms lacking a wild-type Nod2 polypeptide and methods of identifying agents useful to treat inflammatory bowel disease (e.g., Crohn's disease).

### BACKGROUND

Crohn's Disease (CD) is a chronic inflammatory bowel disease (IBD) thought to be caused by genetic and environmental factors that affect host-microbe interactions and production of inflammatory mediators (Girardin et al., Trends Immunol 24, 652-658 (2003); C. Fiocchi, Gastroenterology 115, 182-305 (1998)). Mutations that increase susceptibility to CD by up to 40-fold were mapped to the NOD2/CARD15 locus (Ogura et al., Nature 411, 603-606 (2001); J. P. Hugot et al., Nature 411, 599-603 (2001)). NOD2 protein contains two N-terminal caspase recruitment domains (CARDs), a nucleotide binding domain (NBD), and ten C-terminal leucine rich repeats (LRRs), and is expressed mainly by macrophages and dendritic cells (Y. Ogura et al., J. Biol. Chem. 276, 4812-4818 (2001)). NOD2 mediates intracellular recognition of muramyl dipeptide (MDP), a building block for bacterial cell wall, and can activate NF-κB (Id.). Macrophages within the intestinal lamina propria of CD patients overproduce NF-κB targets, including the proinflammatory cytokines tumor necrosis factor α (TNFα), IL-1β, and IL-6 (Fiocchi et al., supra; Podolsky, N Engl J Med 347, 417-429 (2002)). Many of the anti-inflammatory drugs used to treat CD inhibit NF-κB activation, suggesting it is a key pathogenic factor (Podolsky, supra). However, paradoxically, transient transfection experiments suggest that CD-associated NOD2 variants no longer activate NF-κB in response to muramyl dipeptide (MDP) (Inohara et al., J Biol Chem 278, 5509-5512 (2003); Girardin et al., J Biol Chem 278, 8869-8872 (2003)), suggesting that defective NF-κB activation in macrophages facilitates infection of the lamina propria by enteric bacteria. However, macrophages can activate NF-κB in response to bacteria independently of NOD2 (Kopp et al., Curr Opin Immunol 15, 396-401 (2003)), and Nod2 gene ablation did not cause spontaneous intestinal infections or colonic inflammation (Pauleau et al., Mol Cell Biol 23, 7531-7539 (2003)).

### SUMMARY

The invention provides useful models for studying inflammatory bowel syndrome such as, for example, Crohn's Disease. The invention also provide methods for identifying therapeutics useful in the treatment of inflammatory bowel diseases including Crohn's disease.

The invention provides a method of inducing inflammatory bowel disease (IBD)-like symptoms in an animal, comprising contacting a transgenic non-human animal comprising a mutant Nod2 gene product with an agent that induces IBD-like symptoms.

The invention also provides a method of generating an inflammatory bowel disease animal model, comprising (i) providing an embryonic stem (ES) cell from a relevant animal species comprising a Nod2 gene; (ii) providing a targeting vector comprising a polynucleotide having a mutant Nod2 polynucleotide capable of homologous recombination with the Nod2 gene; (iii) introducing the targeting vector into the ES cells under conditions where the Nod2 gene undergoes homologous recombination with the targeting vector to produce a mutant Nod2 gene; (iv) introducing the ES cells carrying a mutant Nod2 gene into a blastocyst; (v) implanting the blastocyst into the uterus of pseudopregnant female; (vi) delivering animals from said female; and (vii) selecting for transgenic Nod2 mutant animals. In one aspect, the animal model is a mouse model. Also provided is a transgenic non-human animal produced by the foregoing method.

The invention provides a transgenic non-human animal comprising a mutant Nod2 gene, wherein the transgenic non-human animal demonstrates a phenotype, when contacted with muramyl dipeptide (MDP), of increased activation of NF-κB and/or increased interleukin-1β secretion. In yet another aspect, the transgenic non-human animal is a Nod2^{2939iC} transgenic mouse.

The invention also provides primary cells and cell lines derived from a transgenic non-human animal of the invention as described herein. In one aspect, the primary cells or cell lines are derived from bone marrow of the transgenic non-human animal. In another aspect, the cell line is a bone marrow derived macrophage cell line. In yet a further aspect, the cell line is an intestinal epithelial cell line.

The invention provides a method of screening an agent for its efficacy in ameliorating the symptoms of inflammatory bowel disease (IBD), comprising administering a candidate agent to a non-human transgenic animal comprising a mutated Nod2 gene product, wherein the non-human transgenic animal is characterized by having elevated interleukin-1β levels when contacted with MDP; and comparing the symptoms of IBD in the non-human transgenic animal to one or more control animals, wherein a decrease in symptoms of IBD in the animal treated with the test agent indicates efficacy of the agent.

The invention further provides a method of inhibiting an inflammatory bowel disease (IBD) in a subject having or at risk of having such a disease comprising contacting the subject with an agent that inhibits the activity of an N-terminal CARD domain of a Nod2 polypeptide.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIGS. 1A-E show the generation of Nod2^{2939iC} mice. (A) Schematic structure of NOD2, sequence of WT and mutant alleles around the 2939insC mutation, targeting vector and the targeted locus. Solid boxes—exons, lines—introns. The Neo^{r} cassette was inserted opposite to the Nod2 transcription unit. (B) Southern blot analysis of NcoI-digested genomic DNA from F2 mice of the indicated genotypes. m=mutant allele, +=WT allele. (C) Nod2 mRNA in BMDMs. RNA was converted to cDNA and amplified using primers for 3 different regions of Nod2 cDNA. (D) Expression of WT and truncated (m/m) NOD2 proteins. BMDM lysates were immunoblotted with anti-NOD2 and anti-actin antibodies, to control loading. (E) Shows a targeting vector map used in the invention.

FIGS. 2A-E show Nod2^{2939iC} macrophages exhibit elevated NF-κB activation and IL-1β secretion in response to MDP. (A) BMDMs from WT and Nod2^{2939iC} (m/m) mice were incubated with MDP (1 μg/ml). When indicated, cytosolic and nuclear extracts were prepared and used to analyze IKK activation (KA), IκBα degradation and NF-κB DNA binding activity, respectively. Nuclear extract quality was monitored by measuring nuclear factor-Y (NF-Y) DNA binding. (B) BMDMs were stimulated with Pam₃Cys (1 μg/ml), LPS (100 ng/ml) or CpG DNA (1 μM) to activate TLR2, 4 and 9, respectively. When indicated, nuclear extracts were prepared and NF-κB DNA binding activity was analyzed. (C) Expression of NF-κB target genes was examined in Nod2^{2939iC} and WT macrophages stimulated with MDP, LPS or peptidoglycan (PGN from Staphylocuccus aureus, 10 μg/ml). After 4 hrs cells were collected, total RNA was prepared and gene expression was analyzed by real-time PCR. Data are presented as fold-increase in mRNA expression in Nod2^{2939iC} macrophages relative to WT macrophages, which was given an arbitrary level of 1.0 for each gene. Results are averages ±S.E. of three independent experiments. (D) Elevated IL-1β secretion in MDP-stimulated Nod2^{2939iC} macrophages. WT and Nod2^{2939iC} (m/m) BMDMs were stimulated as indicated. After 24 hrs culture supernatants were collected, and secreted cytokines were measured. (E) MDP induces IL-1β release by Nod2^{2939iC} (m/m) BMDMs. Macrophages were treated with MDP or LPS for 24 hrs. Culture supernatants were collected and analyzed by immunoblotting with anti-IL-1β and anti-TNFα antibodies.

FIGS. 3A-F show enhanced NF-κB activation and inflammation in DSS-treated Nod2^{2939iC} mice. (A) Increased body weight loss in DSS-exposed Nod2^{2939iC} mice. Mice of either genotype were given 3% DSS in drinking water for 6 days and weighted daily. Data are means ±SEM. Asterisks: significant differences (p<0.05). (B) Typical colon appearance (upper panels) and histology (bottom panels) 11 days after initiation of DSS administration. Nod2^{2939iC} mice exhibit more inflammation and ulceration. Arrowheads: borders of ulcers. Magnification: 100×. (C) Induction of inflammation-associated genes in colons of DSS-treated mice. Colonic RNA isolated 11 days after initiation of DSS treatment was analyzed by real-time PCR. Results are averages ±S.E. of fold increase in normalized (relative to GAPDH mRNA) mRNA amounts in DSS-treated mice over untreated mice of same genotype (n=4 per group). (D) Elevated IL-1β and IL-6 in colons of DSS-treated Nod2^{2939iC} mice. The indicated cytokines were measured in colonic extracts prepared 0 or 11 days after DSS exposure. Results are averages ±SD (n=4-8). Asterisk: significant difference (p<0.05). (E) Immunohistochemical detection of IL-6 and Cox-2. Colon sections prepared 11 days after initiation of DSS treatment were analyzed by indirect immunoperoxidase staining for IL-6 and Cox-2. Magnification: 100×. (F) Colonic NF-κB and IKK activities. Nuclear and cytosolic extracts of colonic mucosa prepared 0 and 11 days after initiation of DSS administration were analyzed for NF-κB DNA binding and IKK kinase (KA) activities. Protein recovery in nuclear extracts was determined by immunoblotting with anti-histone deacetylase (HDAC) antibody.

FIGS. 4A-D show that IL-1β is an important contributor in elevated colonic inflammation in Nod2^{2939iC} mice. (A, B) Increased macrophage apoptosis in Nod2^{2939iC} (m/m) mice treated with DSS. Tissue specimens prepared 0 and 11 days after initiation of DSS administration were analyzed by TUNEL staining (A) or by TUNEL plus immunoperoxidase staining for F4/80 (B) Magnification: A—200×; B—400× (C). Increased body weight loss in DSS-exposed Nod2^{2939iC} (mice) is IL-1β dependent. Mice of either genotype were given 3% DSS for 6 days with or without concomitant treatment with IL-1RA (100 mg/kg/day). Mice were weighted daily. Data are means ±SEM. Asterisks: significant differences (WT vs. m/m: p<0.05). (D) Histological inflammation and tissue damage scores were determined 11 days after initiation of DSS treatment in the mice from Panel C. Results are averages ±SEM. Asterisks: significant differences, p<0.05.

FIG. 5 shows the histological appearance of the colon and small intestines of 13-month old Nod2^{2939iC} and WT mice. The tissues (small intestine and colon) were fixed, sectioned and stained with H & E. Magnification: 100×.

FIG. 6 shows activation of JNK, ERK, and p38 by immunoblotting with antibodies that recognize the total MAPK amount or its activated (phosphorylated) form in stimulated BMDMs from WT and Nod2^{2939iC} mice and their cytosolic extracts.

FIG. 7 shows elevated secretion of IL-1β by Nod2^{2939iC} macrophages stimulated with MDP. WT and Nod2^{2939iC} (m/m) BMDMs were stimulated with either LPS, PGN, MDP, Pam₃Cys or PGN+MDP. After 4 or 24 hrs culture supernatants were collected and cytokine levels were measured by ELISA.

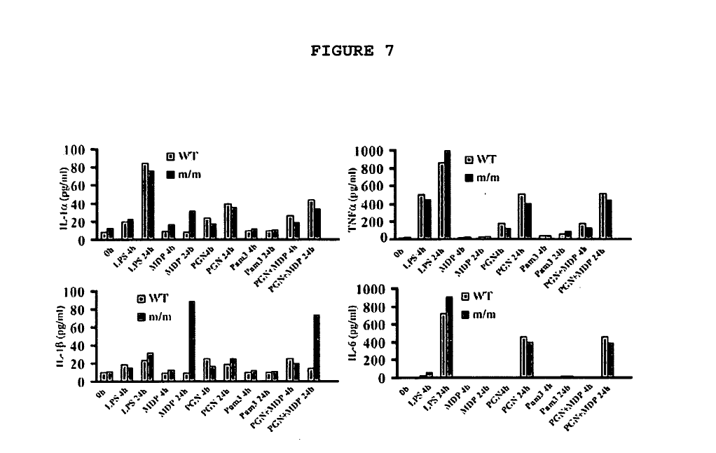

FIG. 8 shows a survival curves of WT (n=19) and Nod2^{2939iC} (n=16) mice treated with DSS (3%) for 6 days. Significantly increased mortality was found in Nod2^{2939iC} mice relative to WT mice (37.5% vs. 0%) by 10 days after DSS exposure.

FIG. 9 shows increased macrophage infiltration into colons of DSS-treated Nod2^{2939iC} mice. Tissue specimens prepared 11 days after initiation of DSS exposure were analyzed by indirect immunoperoxidase staining with anti-F4/80 antibody. Magnification: 200×.

FIG. 10A-B show an increased expression of IL-6, Cox-2 and nuclear RelA in DSS-treated Nod2^{2939iC} mice. (A) IL-6- or Cox-2-positive and nuclear RelA staining cells were counted in areas of the colon showing moderate or severe inflammation 11 days after DSS exposure. Asterisks: significant differences (p<0.05). (B) Typical examples of IL-6 immunostaining in colon sections of DSS-treated mice showing moderate or severe inflammation. Magnification: 200×.

FIG. 11 shows increased RelA nuclear staining in colons of DSS-treated Nod2^{2939iC} mice. Tissue specimens prepared 0 or 11 days after initiation of DSS treatment were analyzed by indirect immunoperoxidase staining with anti-RelA(p65) antibody. Arrowheads indicate positive nuclear staining. Magnification: 400× (left panels) or 600× (right panels).

FIG. 12 shows an analysis of MAPK activation in DSS-treated mice. Cytosolic extracts of colonic mucosa were prepared before or 11 days after initiation of DSS treatment. Total JNK, ERK or p38 MAPK levels were determined by immunoblotting and their activation states were examined using antibodies that specifically recognize their phosphorylated and activated forms. No p38 activation could be detected.

FIGS. 13A-B show antibiotic treatment eliminates genotype-specific differences in the inflammatory response to DSS. (A) Body weight curves of mice receiving DSS plus antibiotics. WT and Nod2^{2939iC} (m/m) mice were given 6% DSS in the drinking water for 6 days together with broad spectrum antibiotics (neomycin sulfate, 1.5 g/L and metronidazole, 1.5 g/L). Mice were weighted daily for 9 days. Data are means ±SEM, (n=6). (B) Histological scores of tissue specimens from WT and m/m mice (n=6) collected at day 11 after initiation of DSS plus antibiotics treatment.

FIG. 14 shows a typical colon histology of WT and Nod2^{2939iC} mice 11 days after initiation of DSS plus IL-1RA (100 mg/kg/day) treatment. The colons of both mice exhibit decreased inflammation and ulceration compared to ones treated with DSS alone (shown in FIG. 3).

FIGS. 15A-B shows a deletion of IKKβ in hematopoietic cells reduces DSS-induced colonic inflammation. To delete Ikkβ in MX1Cre-Ikkβ^{F/F} mice, 2 month old mice were given two injections (250 μl each) of 1 mg/ml poly(IC). Control mice (Ikkβ^{F/F}) were treated similarly. Four days after the last injection, the mice were placed on 2.5% DSS in the drinking water. (A) Histological scores of Ikkβ^{F/F} (F/F) and MX1Cre-Ikkβ^{F/F} (ΔIKKβ) mice (n=4), determined at day 11 after initiation of DSS treatment. The asterisk indicates a significant difference (p<0.05). (B) Typical colon histology of Ikkβ^{F/F} (F/F) and MX1Cre-Ikkβ^{F/F} (ΔIKKβ) mice 11 days after initiation of DSS administration. The colon of ΔIKKβ mice exhibits decreased inflammation and ulceration compared to the colon of F/F mice.

### DETAILED DESCRIPTION

Inflammatory bowel diseases (IBD) are defined by chronic, relapsing intestinal inflammation. IBD includes two disorders, Crohn's disease and ulcerative colitis (UC). Both diseases appear to involve either a dysregulated immune response to GI tract antigens, a mucosal barrier breach, and/or an adverse inflammatory reaction to a persistent intestinal infection. The GI tract luminal contents and bacteria constantly stimulate the mucosal immune system, and a delicate balance of proinflammatory and anti-inflammatory cells and molecules maintains the integrity of the GI tract, without eliciting severe and damaging inflammation. It is unknown how the IBD inflammatory cascade begins, but constant GI antigen-dependent stimulation of the mucosal and systemic immune systems perpetuates the inflammatory cascade and drives lesion formation.

There is no known cure for IBD. In subjects with IBD, the inner lining of the intestines is afflicted with ulcers and inflammation which lead to symptoms of abdominal pain, diarrhea, and rectal bleeding. Ulcerative colitis typically occurs in the large intestine, while Crohn's disease typically involves the entire GI tract as well as the small and large intestines. For most subjects afflicted with IBD, the symptoms last for months to years. Common clinical symptoms of IBD are intermittent rectal bleeding, crampy abdominal pain, weight loss and diarrhea. Diagnosis of IBD is based on the clinical symptoms, the use of a barium enema, but direct visualization (sigmoidoscopy or colonoscopy) is the most accurate test. Protracted IBD has been identified as a risk factor for colon cancer.

In subjects with more extensive IBD, blood loss from the inflamed intestines can lead to anemia, and may require treatment with iron supplements or even blood transfusions. Rarely, the colon can acutely dilate to a large size when the inflammation becomes very severe. This condition is called toxic megacolon. Patients with toxic megacolon are extremely ill with fever, abdominal pain and distention, dehydration, and malnutrition. Unless a subject improves rapidly with medication, surgery is usually necessary to prevent colon rupture.

Crohn's disease can occur in all regions of the gastrointestinal tract. With this disease intestinal obstruction due to inflammation and fibrosis occurs in a large number of subjects. Granulomas and fistula formation are frequent complications of Crohn's disease. Disease progression consequences include intravenous feeding, surgery and colostomy.

The most commonly used medications to treat IBD are anti-inflammatory drugs. For example, both salicylates and corticosteroids are commonly used, but both have side effects. In IBD patients that do not respond to salicylates or corticosteroids, medications that suppress the immune system are used. Examples of immunosuppressants include azathioprine and 6-mercaptopurine. Immunosuppressants used in this situation help to control IBD and allow gradual reduction or elimination of corticosteroids. However, immunosuppressants cause increased risk of infection, renal insufficiency, and the need for hospitalization.

Increasing evidence implicates mutations in a family of proteins that regulate innate immune responses resulting in pathogenic infections. This family of cytoplasmic proteins, collectively termed Nod, is characterized by the presence of three motifs: a CARD, an NBD (nucleotide binding domain) and an LRR. These proteins have homology to the NBD-LRR type disease resistant gene products in plants. An increasing number of the members of this family have been identified (Nod1/CARD4, Nod2, DEFCAP/NAC, CARD12/Ipaf/CLAN) and by analogy to the plant molecules these data imply that Nod proteins are a diverse family of molecules designed to detect pathogens in intracellular compartments; the LRR of members of both families is likely to confer pathogen specificity. In fact, Nod1 is activated upon infection of Shigella flexneri in epithelial cells and one NBD-LRR protein, NAIP determines susceptibility to Legionella pneumophila infection.

Nod proteins belong to the NBS-LRR protein (for nucleotide-binding site and leucine-rich repeat) family, which are involved in intracellular recognition of microbes and their products. NBS-LRR proteins are characterized by three domains: a C-terminal leucine-rich repeat (LRR) domain able to sense a microbial motif, an intermediary nucleotide binding site (NBS) essential for the oligomerization of the molecule that is necessary for the signal transduction induced by different N-terminal effector motifs, such as a caspase-activating and recruitment domain (CARD). Nod1 and Nod2 comprise these domains and play a role in the regulation of pro-inflammatory pathways through NF-κB induced by bacterial motifs. For example, Nod2 recognizes muramyl dipeptide (MDP), a specific peptidoglycan motif from bacteria. A number of genetic disorders have been linked to NBS-LRR proteins. For example, mutations in Nod2, are associated with susceptibility to a chronic intestinal inflammatory disorder, Crohn's disease. Mutations in the NBS region of Nod2 induce a constitutive activation of NF-κB and are responsible for Blau syndrome (Chamaillard et al., Cellular Microbiology, 5(9):581-592, 2003).

It has recently been shown that variants of Nod2, an intracellular sensor of bacterial-derived muramyl dipeptide (MDP), increase susceptibility to Crohn's Disease (CD) and Blau's syndrome. Three main (two missense and one frameshift) Nod2 mutations associated with Crohn's disease have been identified; each alters the structure of either the LRR domain or the adjacent region of the protein. Thus, the LRR domain of the Crohn's disease-associated variants is likely to be impaired in its recognition of microbial components. Furthermore, these variants are thought to be defective in activation of nuclear factor—kappaB (NF-κB) and antibacterial defenses, but CD clinical specimens display elevated NF-κB activity.

The production of interleukin-1β (IL-1β), a pro-inflammatory cytokine, has been demonstrated to be mediated by activated caspase-1. A molecular mechanisms underlying caspase-1 processing and activation involves interaction between the caspase recruit domains (CARDs) of caspase-1 and a serine/threonine kinase RIP2. Nod1 and 2 are suspected of playing a role in the association of both caspase-1 and RIP2. Nod1 and 2 thus play a role in caspase-1 activation and IL-1β processing (Yoo et al., Biochem Biophys Res. Comm., 299(4):652-658, 2002).

Nod1 and 2 polypeptide and polynucleotide sequences are known (see, e.g., U.S. Pat. No. 6,858,391, the disclosure of which is incorporated herein by reference in its entirety). For example, a sequence of Nod1 is available on GenBank as accession No. AF 113925, AC007728 and AQ534686. The genomic sequence of Nod2 is available as GenBank accession numbers AC007728 and AC007608 and the cDNA sequence as GenBank accession No. AF178930 and AH012203. Homologs from other organisms can be identified based upon sequence identity. The above identified GenBank references are incorporated herein by reference in the entirety.

The availability of molecular clones for the Nod family of proteins has enabled the rapid (and continuing) functional characterization of these polypeptides. Although cloning of Nod polypeptides is a first step to understanding their functions, such in vitro and in silico studies do not provide a full understanding of a polypeptide's function. In vivo functional analysis can be achieved by gene knockout techniques in mammalian systems (e.g., in mice, rats, and the like). The direct approach to elucidation of the in vivo function of the Nod family of proteins is of course through generation of the corresponding knockout organisms. Thus, the invention provides knockout non-human organisms lacking one or more Nod genes (e.g. Nod2).

The invention provides a model of IBD including Crohn's disease and/or Blau syndrome. Furthermore, the invention provides methods and compositions useful to identify agents that are capable of treating Crohn's disease and/or Blau syndrome. To illuminate the pathophysiological function of Nod2, variant(s) of Nod2 were introduced into a mouse Nod2 locus. Transgenic mutant mice exhibited elevated NF-κB activation in response to MDP and more efficient processing and secretion of the cytokine interleukin-1β (IL-1β). These effects are linked to increased susceptibility to bacterial-induced intestinal inflammation and identify Nod2 as a positive regulator of NF-κB activation and IL-1β secretion.

The invention provides transgenic animals comprising an exogenous Nod2 gene or homologs, mutants, or variants thereof. The non-human transgenic animals of the invention display an altered phenotype as compared to wild-type animals. In one embodiment, the altered phenotype is the decreased expression of mRNA encoding a functional Nod2 polypeptide compared to wild-type levels of endogenous Nod2 expression. Methods for analyzing the presence or absence of such phenotypes include Northern blotting, mRNA protection assays, and RT-PCR. In another embodiment, the non-human transgenic animal comprises a knockout mutation of the Nod2 gene. In yet another embodiment, expression of a Nod2 variant gene (e.g., a Nod2 polynucleotide sequence comprising 5′-TACCGGGGTGCAGAAGCCCTCCTGCAGGCCCCATGA-3′ (SEQ ID NO:1)), which comprises a single nucleotide insertion compared to the wild-type Nod2, variants or mutants containing deletions of one or more LRR repeats is also encompassed by the transgenic non-human animal. In a further aspect, the transgenic non-human animal comprises a mutation in the Nod2 locus such that the animal expresses a Nod2 comprising a missense or frameshift mutation associated with IBD in the human homolog. In another aspect, such non-human transgenic organisms display a phenotype and symptoms associated with IBD including Crohn's disease.

The non-human transgenic organisms of the invention find use in pathogen (e.g., enteric bacteria) screens, dietary and drug screening. For example, the transgenic organisms (e.g., displaying a Crohn's disease phenotype) are fed a test agent (e.g., drugs, dietary agents, pathogens) and the response of the organism to the agent(s) is evaluated. Such screening will utilize proper use of controls (e.g., placebos) and the control organism are then compared to the results from treated organisms. In another example, transgenic and control organisms are treated with an agent that induces susceptibility to IBD and/or are infected with a pathogen (e.g., bacteria) found to cause or increase the severity of disease symptoms, followed by the administration of test agent and control agent. The effects of the test and control agents on disease symptoms are then assessed.

“Transgenic organism” refers to an animal in which exogenous DNA has been introduced while the animal is still in its embryonic stage. In most cases, the transgenic approach aims at specific modifications of the genome, e.g., by introducing whole transcriptional units into the genome, or by up- or down-regulating or mutating pre-existing cellular genes. The targeted character of certain of these procedures sets transgenic technologies apart from experimental methods in which random mutations are conferred to the germline, such as administration of chemical mutagens or treatment with ionizing solution. A transgenic organism can include an organism which has a gene knockout or may result for inducing a genetic mutation.

“Knockout” refers to partial or complete suppression of the expression of a protein encoded by an endogenous DNA sequence in a cell. The “knockout” can be affected by targeted deletion of the whole or part of a gene encoding a protein. Alternatively, the transgenic organism can be obtained by the targeted mutation of a functional protein in an embryonic stem cell. As a result, the deletion or mutation may prevent or reduce the expression of the protein in any cell in the whole animal in which it is normally expressed, or results in the expression of a mutant protein having biological function different than the normal/wild-type protein. For example, a “Nod2 transgenic animal” refers to an animal in which the expression of Nod2 has been reduced or suppressed by the introduction of a recombinant nucleic acid molecule that disrupts at least a portion of the genomic DNA sequence encoding Nod2 or mutates a Nod2 genetic sequence such that the resulting expressed polypeptide is mutated.

The term “knockout animal,” “transgenic animal” and the like, refers to a transgenic animal wherein a given gene has been suppressed or mutated by recombination with a targeting vector. It is to be emphasized that the term is intended to include all progeny generations. Thus, the founder animal and all F1, F2, F3, and so on, progeny thereof are included.

The term “chimera,” “mosaic,” “chimeric mammal” and the like, refers to a transgenic mammal with a knockout or mutation in some of its genome-containing cells.

The term “heterozygote,” “heterozygotic mammal” and the like, refers to a transgenic mammal with a knockout or mutation on one of a chromosome pair in all of its genome-containing cells.

The term “homozygote,” “homozygotic mammal” and the like, refers to a transgenic mammal with a knockout or mutation on both members of a chromosome pair in all of its genome-containing cells.

A “non-human animal” of the invention includes mammals such as rodents, non-human primates, sheep, dog, cow, chickens, amphibians, reptiles, etc. Typical non-human animals are selected from the rodent family including rat and mouse, most typically mouse, though transgenic amphibians, such as members of the Xenopus genus, and transgenic chickens can also provide important tools for understanding and identifying agents which can affect, for example, protein function and disease models.

A “mutation” is a detectable change in the genetic material in the animal, which is transmitted to the animal's progeny. A mutation is usually a change in one or more deoxyribonucleotides, the modification being obtained by, for example, adding, deleting, inverting, or substituting nucleotides.

Typically, the genome of the transgenic non-human mammal comprises one or more deletions in one or more exons of the genes and further comprises a heterologous selectable marker gene.

In principle, transgenic animals may have one or both copies of the gene sequence of interest disrupted or mutated. In the case where only one copy of the nucleic acid sequence of interest is disrupted or mutated, the knockout animal is termed a “heterozygous transgenic organism”.

It is important to note that it is not necessary to disrupt a gene to generate a transgenic organism lacking functional expression. The invention includes the use of antisense molecules that are transformed into a cell, such that production of a Nod1 and/or 2 polypeptide is inhibited. Such an antisense molecule is incorporated into a germ cell as described more fully herein operably linked to a promoter such that the antisense construct is expressed in all cells of a transgenic organism.

The techniques for introducing foreign DNA sequences into the mammalian germ line were originally developed in mice. One route of introducing foreign DNA into a germ line entails the direct microinjection of linear DNA molecules into a pronucleus of a fertilized one-cell egg. Microinjected eggs are subsequently transferred into the oviducts of pseudopregnant foster mothers and allowed to develop. About 25% of the progeny mice inherit one or more copies of the micro-injected DNA. Currently, the most frequently used techniques for generating chimeric and transgenic animals are based on genetically altered embryonic stem cells or embryonic germ cells. A suitable technique for obtaining completely ES cell derived transgenic non-human organisms is described in WO 98/06834.

In another aspect, embryonic stem cell mutants/knockouts are used to obtain the transgenic organism (e.g., a Nod1 and/or Nod2 mutant/knockout transgenic organism). Thus, the invention relates to a method for producing a Nod2 transgenic non-human organism comprising (i) providing an embryonic stem (ES) cell from the relevant organism species comprising an intact Nod2) gene; (ii) providing a targeting vector capable of disrupting or mutating the intact Nod2 gene; (iii) introducing the targeting vector into the ES cells under conditions where the intact Nod2 gene undergoes homologous recombination with the targeting vector to produce a mutant Nod2 gene; (iv) introducing the ES cells carrying a mutated or disrupted Nod2 gene into a blastocyst; (v) implanting the blastocyst into the uterus of pseudopregnant female; and (vi) delivering animals from said females, and breeding them.

Transgenic mutant or knockout mice are generated by homologous integration of a “targeting vector” construct into a mouse embryonic stem cell chromosome which encodes a gene to be knocked out or mutated. In one embodiment, gene targeting, which is a method of using homologous recombination to modify an animal's genome, can be used to introduce changes into cultured embryonic stem cells. By targeting a Nod2 gene of interest in ES cells, these changes can be introduced into the germlines of animals to generate chimeras. The gene targeting procedure is accomplished by introducing into tissue culture cells a DNA targeting vector that includes a segment homologous to a target Nod2 locus, and which also includes an intended sequence modification to the Nod2 genomic sequence (e.g., insertion, deletion, point mutation). The treated cells are then screened for accurate targeting to identify and isolate those which have been properly targeted.

A “targeting vector” is a vector comprising sequences that can be inserted into a Nod2 gene to be disrupted, e.g., by homologous recombination. The targeting vector generally has a 5′ flanking region and a 3′ flanking region homologous to segments of the gene of interest, surrounding a foreign DNA sequence to be inserted into the gene. For example, the foreign DNA sequence may encode a selectable marker, such as an antibiotics resistance gene. Examples for suitable selectable markers are the neomycin resistance gene (NEO) and the hygromycin β-phosphotransferase gene. The 5′ flanking region and the 3′ flanking region are homologous to regions within the gene surrounding the portion of the gene to be replaced with the unrelated DNA sequence. DNA comprising the targeting vector and the native gene of interest are contacted under conditions that favor homologous recombination. For example, the targeting vector and native gene sequence of interest can be used to transform embryonic stem (ES) cells, in which they can subsequently undergo homologous recombination.

Thus, a targeting vector refers to a nucleic acid that can be used to decrease, suppress, or mutate expression of a protein encoded by endogenous DNA sequences in a cell. In a simple example, the targeting vector (sometimes referred to as a knockout construct) is comprised of a 1 kb fragment of Nod2 DNA containing a portion of mutated exon 11 upstream of a neomycin resistance (Neo^{r}) gene, and a 3 kb fragment of Nod2 DNA containing the remainder of exon 11, the intron and exon 12 immediately downstream. In a further aspect, the targeting vector/construct can comprise a negative selectable marker such as diphtheria toxin (DTA) gene. The resulting construct recombines with the endogenous Nod2 gene to obtain a mutated Nod2 gene with a mutation in a critical portion of the polynucleotide so that a functional Nod2 cannot be expressed therefrom. Alternatively, a number of termination codons can be added to the native polynucleotide to cause early termination of the protein or an intron junction can be inactivated. In a typical targeting vector/construct, some portion of the polynucleotide is replaced with a selectable marker (such as the neo gene).

Proper homologous recombination can be confirmed by Southern blot analysis of restriction endonuclease digested DNA using, as a probe, a non-disrupted region of the gene. Since the native gene will exhibit a restriction pattern different from that of the disrupted gene, the presence of a disrupted gene can be determined from the size of the restriction fragments that hybridize to the probe.

In an animal obtained by the methods above, the extent of the contribution of the ES cells that contain the disrupted/mutated Nod2 gene to the somatic tissues of the transgenic animal can be determined visually by choosing animal strains for a source of the ES cells and blastocyst that have different coat colors.

Generally, the embryonic stem cells (ES cells) used to produce the transgenic animals will be of the same species as the knockout animal to be generated. Thus for example, mouse embryonic stem cells will usually be used for generation of knockout mice.

Embryonic stem cells are generated and maintained using methods well known to the skilled artisan such as those described by Doetschman et al. (1985) J. Embryol. Exp. Mol. Biol. 87:27-45). Any line of ES cells can be used, however, the line chosen is typically selected for the ability of the cells to integrate into and become part of the germ line of a developing embryo so as to create germ line transmission of the transgenic/knockout construct. Thus, any ES cell line that is believed to have this capability is suitable for use herein. One mouse strain that is typically used for production of ES cells, is the 129J strain. Another ES cell line is murine cell line D3 (American Type Culture Collection, catalog no. CKL 1934). Still another ES cell line is the WW6 cell line (Ioffe et al. (1995) PNAS 92:7357-7361). The cells are cultured and prepared for knockout construct insertion using methods well known to the skilled artisan, such as those set forth by Robertson in: Teratocarcinomas and Embryonic Stem Cells: A Practical Approach, E. J. Robertson, ed. IRL Press, Washington, D.C. (1987)); by Bradley et al. (1986) Current Topics in Devel. Biol. 20:357-371); and by Hogan et al. (Manipulating the Mouse Embryo: A Laboratory Manual, Cold Spring Harbor Laboratory Press, Cold Spring Harbor, N.Y. (1986)).

Variations on the basic technique described above also exist and are well known in the art. For example, a “knock-in” construct refers to the same basic arrangement of a nucleic acid encoding a 5′ genomic locus fragment linked to nucleic acid encoding a positive selectable marker which in turn is linked to a nucleic acid encoding a 3′ genomic locus fragment, but which differs in that none of the coding sequence is omitted and thus the 5′ and the 3′ genomic fragments used were initially contiguous before being disrupted by the introduction of the nucleic acid encoding the positive selectable marker gene. This “knock-in” type of construct is thus very useful for the construction of mutant transgenic animals when only a limited region of the genomic locus of the gene to be mutated, such as a single exon, is available for cloning and genetic manipulation. Alternatively, the “knock-in” construct can be used to specifically eliminate a single functional domain of the targeted gene, resulting in a transgenic animal which expresses a polypeptide of the targeted gene which is defective in one function, while retaining the function of other domains of the encoded polypeptide. This type of “knock-in” mutant frequently has the characteristic of a so-called “dominant negative” mutant because, especially in the case of proteins which homomultimerize, it can specifically block the action of (or “poison”) the polypeptide product of the wild-type gene from which it was derived. In a variation of the knock-in technique, a marker gene is integrated at the genomic locus of interest such that expression of the marker gene comes under the control of the transcriptional regulatory elements of the targeted gene. One skilled in the art will be familiar with useful markers and the means for detecting their presence in a given cell.

As mentioned above, the homologous recombination of the above described “knockout” and “knock in” constructs is sometimes rare and such a construct can insert nonhomologously into a random region of the genome where it has no effect on the gene which has been targeted for deletion, and where it can potentially recombine so as to disrupt another gene which was otherwise not intended to be altered. Such non-homologous recombination events can be selected against by modifying the above-mentioned targeting vectors so that they are flanked by negative selectable markers at either end (particularly through the use of the diphtheria toxin gene, thymidine kinase gene, the polypeptide product of which can be selected against in expressing cell lines in an appropriate tissue culture medium well known in the art—e.g. one containing a drug such as 5-bromodeoxyuridine. Non-homologous recombination between the resulting targeting vector comprising the negative selectable marker and the genome will usually result in the stable integration of one or both of these negative selectable marker genes and hence cells which have undergone non-homologous recombination can be selected against by growth in the appropriate selective media (e.g. media containing a drug such as 5-bromodeoxyuridine). Simultaneous selection for the positive selectable marker and against the negative selectable marker will result in a vast enrichment for clones in which the construct has recombined homologously at the locus of the gene intended to be mutated. The presence of the predicted chromosomal alteration at the targeted gene locus in the resulting stem cell line can be confirmed by means of Southern blot analytical techniques which are well known to those familiar in the art. Alternatively, PCR can be used.

Each targeting vector to be inserted into the cell is linearized. Linearization is accomplished by digesting the DNA with a suitable restriction endonuclease selected to cut only within the vector sequence and not the 5′ or 3′ homologous regions or the selectable marker region.

For insertion, the targeting vector is added to the ES cells under appropriate conditions for the insertion method chosen, as is known to the skilled artisan. For example, if the ES cells are to be electroporated, the ES cells and targeting vector are exposed to an electric pulse using an electroporation machine and following the manufacturer's guidelines for use. After electroporation, the ES cells are typically allowed to recover under suitable incubation conditions. The cells are then screened for the presence of the targeting vector as explained herein. Where more than one construct is to be introduced into the ES cell, each targeting vector can be introduced simultaneously or one at a time.

After suitable ES cells containing the knockout construct in the proper location have been identified by the selection techniques outlined above, the cells can be inserted into an embryo. Insertion may be accomplished in a variety of ways known to the skilled artisan, however the typical method is by microinjection. For microinjection, about 10-30 cells are collected into a micropipet and injected into embryos that are at the proper stage of development to permit integration of the foreign ES cell containing the recombination construct into the developing embryo. For instance, the transformed ES cells can be microinjected into blastocytes. The suitable stage of development for the embryo used for insertion of ES cells is very species dependent, however for mice it is about 3.5 days. The embryos are obtained by perfusing the uterus of pregnant females. Suitable methods for accomplishing this are known to the skilled artisan.

While any embryo of the right stage of development is suitable for use, typical embryos are male. In mice, the typical embryos also have genes coding for a coat color that is different from the coat color encoded by the ES cell genes. In this way, the offspring can be screened easily for the presence of the knockout construct by looking for mosaic coat color (indicating that the ES cell was incorporated into the developing embryo). Thus, for example, if the ES cell line carries the genes for white fur, the embryo selected will carry genes for black or brown fur.

After the ES cell has been introduced into the embryo, the embryo may be implanted into the uterus of a pseudopregnant foster mother for gestation. While any foster mother may be used, the foster mother is typically selected for her ability to breed and reproduce well, and for her ability to care for the young. Such foster mothers are typically prepared by mating with vasectomized males of the same species. The stage of the pseudopregnant foster mother is important for successful implantation, and it is species dependent. For mice, this stage is about 2-3 days pseudopregnant.

Offspring that are born to the foster mother may be screened initially for mosaic coat color where the coat color selection strategy has been employed. In addition, or as an alternative, DNA from tail tissue of the offspring may be screened for the presence of the construct nucleic acid sequences using Southern blots and/or PCR. Offspring that appear to be mosaics may then be crossed to each other, if they are believed to carry the construct in their germ line, in order to generate homozygous mutant or knockout animals. Homozygotes may be identified by Southern blotting of equivalent amounts of genomic DNA from mice that are the product of this cross, as well as mice that are known heterozygotes and wild type mice.

Other means of identifying and characterizing the transgenic offspring are available. For example, Northern blots can be used to probe the mRNA for the presence or absence of transcripts encoding either the gene knocked out or mutated, the marker gene, or both. In addition, Western blots can be used to assess the level of expression of the Nod2 gene that is mutated or knocked out in various tissues of the offspring by probing the Western blot with an antibody against the particular Nod2 protein or domain, or an antibody against the marker gene product, where this gene is expressed. Finally, in situ analysis (such as fixing the cells and labeling with antibody) and/or FACS (fluorescence activated cell sorting) analysis of various cells from the offspring can be conducted using suitable antibodies to look for the presence or absence of the knockout construct gene product.

Other methods of making transgenic animals are also generally known. See, for example, Manipulating the Mouse Embryo, (Cold Spring Harbor Laboratory Press, Cold Spring Harbor, N.Y., 1986). Recombinase dependent transgenic organisms can also be generated, e.g. by homologous recombination to insert target sequences, such that tissue specific and/or temporal control of inactivation of a Nod2 gene can be controlled by recombinase sequences.

Animals containing more than one transgenic construct and/or more than one transgene expression construct are prepared in any of several ways. A typical manner of preparation is to generate a series of animals, each containing one of the desired transgenic phenotypes. Such animals are bred together through a series of crosses, backcrosses and selections, to ultimately generate a single animal containing all desired transgenic traits and/or expression constructs, where the animal is otherwise congenic (genetically identical) to the wild type except for the presence of the construct(s) and/or transgene(s).

In another aspect, a transgenic animal can be obtained by introducing into a single stage embryo a targeting vector. The zygote is the best target for micro-injection. In the mouse, the male pronucleus reaches the size of approximately 20 micrometers in diameter which allows reproducible injection of 1-2 pl of DNA solution. The use of zygotes as a target for gene transfer has an advantage in that in most cases the injected DNA will be incorporated into the host gene before the first cleavage (Brinster et al. (1985) PNAS 82:4438-4442). As a consequence, all cells of the transgenic animal will carry the incorporated nucleic acids of the targeting vector. This will in general also be reflected in the efficient transmission to offspring of the founder since 50% of the germ cells will harbor the transgene.

Normally, fertilized embryos are incubated in suitable media until the pronuclei appear. At about this time, the nucleotide sequence comprising the transgene is introduced into the female or male pronucleus. In some species such as mice, the male pronucleus is typically used. Typically the exogenous genetic material is added to the male DNA complement of the zygote prior to its being processed by the ovum nucleus or the zygote female pronucleus. It is thought that the ovum nucleus or female pronucleus release molecules which may affect the male DNA complement, perhaps by replacing the protamines of the male DNA with histones, thereby facilitating the combination of the female and male DNA complements to form the diploid zygote.

Thus, the exogenous genetic material is typically added to the male complement of DNA or any other complement of DNA prior to its being affected by the female pronucleus. For example, the exogenous genetic material is added to the early male pronucleus, as soon as possible after the formation of the male pronucleus, which is when the male and female pronuclei are well separated and both are located close to the cell membrane. Alternatively, the exogenous genetic material could be added to the nucleus of the sperm after it has been induced to undergo decondensation. Sperm containing the exogenous genetic material can then be added to the ovum or the decondensed sperm could be added to the ovum with the transgene constructs being added as soon as possible thereafter.

Introduction of the exogenous nucleic acid (e.g., a targeting vector) into the embryo may be accomplished by any means known in the art such as, for example, microinjection, electroporation, or lipofection. Following introduction of the exogenous nucleic acid into the embryo, the embryo may be incubated in vitro for varying amounts of time, or reimplanted into the surrogate host, or both. In vitro incubation to maturity is within the scope of this invention. One common method in to incubate the embryos in vitro for about 1-7 days, depending on the species, and then reimplant them into the surrogate host.

For the purposes of this invention a zygote is essentially the formation of a diploid cell which is capable of developing into a complete organism. Generally, the zygote will be comprised of an egg containing a nucleus formed, either naturally or artificially, by the fusion of two haploid nuclei from a gamete or gametes. Thus, the gamete nuclei must be ones which are naturally compatible, i.e., ones which result in a viable zygote capable of undergoing differentiation and developing into a functioning organism. Generally, a euploid zygote is used. If an aneuploid zygote is obtained, then the number of chromosomes should not vary by more than one with respect to the euploid number of the organism from which either gamete originated.

In addition to biological considerations, physical ones also govern the amount (e.g., volume) of exogenous genetic material which can be added to the nucleus of the zygote or to the genetic material which forms a part of the zygote nucleus. If no genetic material is removed, then the amount of exogenous genetic material which can be added is limited by the amount which will be absorbed without being physically disruptive. Generally, the volume of exogenous genetic material inserted will not exceed about 10 picoliters. The physical effects of addition must not be so great as to physically destroy the viability of the zygote. The biological limit of the number and variety of DNA will vary depending upon the particular zygote and functions of the exogenous genetic material and will be readily apparent to one skilled in the art, because the genetic material, including the exogenous genetic material, of the resulting zygote must be biologically capable of initiating and maintaining the differentiation and development of the zygote into a functional organism.

The number of copies of a transgene (e.g., the exogenous genetic material or targeting vector constructs) which are added to the zygote is dependent upon the total amount of exogenous genetic material added and will be the amount which enables the genetic transformation to occur. Theoretically only one copy is required; however, generally, numerous copies are utilized, for example, 1,000-20,000 copies of a targeting vector construct, in order to insure that one copy is functional.

Reimplantation is accomplished using standard methods. Usually, the surrogate host is anesthetized, and the embryos are inserted into the oviduct. The number of embryos implanted into a particular host will vary by species, but will usually be comparable to the number of offspring the species naturally produces.

Transgenic offspring of the surrogate host may be screened for the presence and/or expression of an exogenous polynucleotide (e.g., that of a targeting vector) by any suitable method as described herein. Alternative or additional methods include biochemical assays such as enzyme and/or immunological assays, histological stains for particular marker or enzyme activities, flow cytometric analysis, and the like.

Progeny of the transgenic animals may be obtained by mating the transgenic animal with a suitable partner, or by in vitro fertilization of eggs and/or sperm obtained from the transgenic animal. Where mating with a partner is to be performed, the partner may or may not be transgenic and/or a knockout. Alternatively, the partner may be a parental line. Where in vitro fertilization is used, the fertilized embryo may be implanted into a surrogate host or incubated in vitro, or both. Using either method, the progeny may be evaluated using methods described above, or other appropriate methods.

Retroviral infection can also be used to introduce a targeting vector into an animal. The developing non-human embryo can be cultured in vitro to the blastocyst stage. During this time, the blastomeres can be targets for retroviral infection (Jaenich, R. (1976) PNAS 73:1260-1264). Efficient infection of the blastomeres is obtained by enzymatic treatment to remove the zona pellucida (Manipulating the Mouse Embryo, Hogan eds. (Cold Spring Harbor Laboratory Press, Cold Spring Harbor, 1986). The viral vector system used to introduce the targeting vector is typically a replication-defective retrovirus carrying the exogenous nucleic acid (Jahner et al. (1985) PNAS 82:6927-6931; Van der Putten et al. (1985) PNAS 82:6148-6152). Transfection is easily and efficiently obtained by culturing the blastomeres on a monolayer of virus-producing cells (Van der Putten, supra; Stewart et al. (1987) EMBO J. 6:383-388). Alternatively, infection can be performed at a later stage. Virus or virus-producing cells can be injected into the blastocoele (Jahner et al. (1982) Nature 298:623-628). Most of the founders will be mosaic for the targeting vector (e.g., the exogenous nucleic acids) since incorporation occurs only in a subset of the cells which formed the transgenic non-human animal. Further, the founder may contain various retroviral insertions of the transgene at different positions in the genome which generally will segregate in the offspring. In addition, it is also possible to introduce transgenes into the germ line by intrauterine retroviral infection of the midgestation embryo (Jahner et al. (1982) supra).

In another aspect, the invention relates to the use of a Nod2 mutant transgenic and/or knockout animal, in particular a mouse, as a model to study inflammatory bowel disease, Crohn's disease, bacterial infection and/or drug therapy. In a further embodiment, the invention relates to cells and tissues that carry mutations in at least one Nod2 gene (e.g., Nod2). The cells can be primary cells or established cell lines obtained from the transgenic animals of the invention according to routine methods, i.e. by isolating and disintegrating tissue, in particular gastrointestinal tissue (e.g., stomach, intestine and the like) and bone marrow derived macrophages are useful. Such cells are harvested from the transgenic animal and passaged appropriately. Such cells and tissues derived from the animals of the invention are useful in in vitro methods relating to the study of inflammation, inflammatory cytokine production, caspase activity, Crohn's disease, inflammatory bowel disease, Blau syndrome, bacterial infection and in the identification of drug candidates.

In a further aspect, the invention relates to a method for determining whether an agent has therapeutic potential in inflammatory bowel disease and/or Crohn's disease, wherein a candidate agent is administered, for example, to a Nod2 transgenic animal and the ability of the agent to ameliorate or reduce one or more symptoms of IBD or Crohn's disease are analyzed.

The test agent can be administered to the non-human transgenic animal in a variety of ways, e.g. orally, in a suitable formulation, by parenteral injection, subcutaneous, intramuscular, or intra-abdominal injection, infusion, ingestion, suppository administration, and skin-patch application. The effect of the agent on, for example, bacterial infection, gastrointestinal lesions, diarrhea, rectal bleeding and the like can be determined using methods well known to a person of ordinary skill in the art.

In an alternative method for screening agents the test agents can be contacted with cells derived from such transgenic animals. In such methods, cells are incubated with the agent. The effect on NF-κB and/or IL-1β expression can then be analyzed on the cellular level to identify agents that effect expression compared to controls.

In one aspect the transgenic animals of the invention provides an animal model for studying the pathophysiology of Inflammatory Bowel Disease and/or Crohn's disease. The model comprises a transgenic mouse whose genome contains a disruption or mutation to a Nod2. In one specific aspect, the transgenic animal comprises a homozygous mutation to Nod2 resulting in a transgenic organism that has elevated NF-κB activation in response to muramyl dipeptide (MDP) and elevated secretion of the cytokine interleukin-1β. A transgenic animal of the invention displays at least one sign or symptom associated with Crohn's disease selected from the group consisting of, for example, the elevated activation of NF-κB, the increase secretion of IL-1β, the increase secretion of tumor necrosis factor alpha (TNFα), abdominal pain, diarrhea, rectal bleeding, Granulomas and fistula.

The invention provides a method of screening a candidate agent for its efficacy in ameliorating the symptoms of IBD. The method comprising administering a candidate agent to a non-human transgenic animal not expressing a wild-type Nod2 gene product, wherein the non-human transgenic animal is characterized by having elevated interleukin-1β levels when contacted with MDP; and comparing the symptoms of IBD in the non-human transgenic animal to one or more control animals (e.g., a non-human transgenic animal that did not receive the test agent, wherein a decrease in symptoms of IBD in the animal treated with the test agent indicates efficacy of the compound. In one aspect the IBD comprises symptoms of Crohn's disease. In another aspect, the non-human transgenic animal comprises a mutation in Nod2, wherein the mutation results in an early termination and C-terminal truncation of the Nod2 polypeptide. The test agent can be any agent suspected of having the ability to treat IBD. Such agents are selected from the group consisting of small molecules, peptides, polypeptides, proteins, peptidomimetics, antibodies, nucleic acids, antisense nucleic acids, ribozymes and the like. In one aspect, the agent inhibits the interaction of a CARD domain of a Nod2 polypeptide with its ligand (e.g., a caspase). In yet another aspect, the agent is an antibody that interacts with a CARD domain.

The invention also provides a method of screening a candidate agent for its efficacy in preventing or delaying the development of IBD. The method comprising administering a candidate agent to a non-human transgenic animal not expressing a wild-type Nod gene product (e.g., a mutated Nod2 gene product), wherein the non-human transgenic animal does not display any symptoms of IBD; the non-human transgenic animal being capable of displaying symptoms of IBD when contacted with MDP, wherein when the transgenic animal is contacted with an agent that induces IBD symptoms, such symptoms comprise elevated interleukin-1β levels. Contacting the animal treated with the test agent with an agent (e.g., MDP) that induces IBD symptoms and comparing the symptoms of IBD in the non-human transgenic animal to one or more control animals (e.g., a non-human transgenic animal that did not receive the test agent), wherein a decrease in symptoms of IBD in the animal treated with the test agent indicates efficacy of the compound. In one aspect the IBD comprises symptoms of Crohn's disease. In another aspect, the non-human transgenic animal comprises a mutation in Nod2, wherein the mutation results in an early termination and C-terminal truncation of the Nod2 polypeptide. The test agent can be any agent suspected of having the ability to treat IBD. Such agents are selected from the group consisting of small molecules, peptides, polypeptides, proteins, peptidomimetics, antibodies, nucleic acids, antisense nucleic acids, ribozymes and the like. In one aspect, the agent inhibits the interaction of a CARD domain of a Nod2 polypeptide with its ligand (e.g., a caspase). In yet another aspect, the agent is an antibody that interacts with a CARD domain.

The invention further provides a method of screening for genes that may be involved in the pathogenesis of IBD and/or Crohn's disease and therefore may be novel targets for the development of drugs for the treatment of IBD. The method comprises administering an agent that induces IBD symptoms to a non-human transgenic animal not expressing a wild-type Nod gene product (e.g., expressing a mutated Nod2 gene product), wherein the non-human transgenic animal is characterized by having elevated interleukin-1β levels when contacted with MDP; administering the same agent to a control animal that expresses a wild-type Nod2 gene product; making RNA preparations from the intestine and/or bone marrow derived macrophages from both the animals after a desired time interval; and comparing the RNA samples, wherein a RNA which shows a difference in these samples indicates a gene that may be implicated in the pathogenesis of IBD. The comparison of the RNA samples mentioned above can be carried out by expression profiling (e.g., by differential display PCR or subtractive hybridization methods or by microarray analysis).

A further aspect of the invention is a method of preparing a composition, which comprises identifying an agent that is capable of ameliorating the symptoms of IBD by one or more of the method described above using a transgenic organism of the invention. The method includes identifying agents that demonstrate efficacy and formulating the agent with a pharmaceutically acceptable carrier. The agent can be an antibody, small molecule, peptide, polypeptide, protein, peptidomimetic, nucleic acid and the like.

The invention demonstrates that Nod2 mutant transgenic mice exhibited elevated NF-κB activation in response to MDP and more efficient processing and secretion of the cytokine interleukin-1β. These effects are linked to increases susceptibility to bacterial-induced intestinal inflammation and identify Nod2 as a positive regulator of NF-κB activation and IL-1 secretion.

These data indicate a key role for Nod2 in gastrointestinal maintenance, immune system function, and inflammation. The Nod2 mutant transgenic mice are fertile and exhibit no obvious morphological defects, but present a distinct physiological phenotype characteristic of Crohn's disease.

Mutant Nod polypeptides can be characterized by having any number of mutations. For example, a Nod polypeptide may be altered by addition, substitution, or deletions of amino acids in order to modify its activity. For example, amino acids may be deleted to remove or modify the activity of the protein. Typically, deletions will be from 1 to 10 amino acids, 11-20 but typically less than 30% of the total number of amino acids in a Nod polypeptide. While random mutations can be made to a Nod polynucleotide (using random mutagenesis techniques known to those skilled in the art) and the resulting mutant Nod polynucleotide used in a targeting vector to generate a transgenic animal. Alternatively, site-directed mutation of a Nod polynucleotide can be engineered (using site-directed mutagenesis techniques well known to those skilled in the art) to create mutant Nod polynucleotide. For example, one can mutate a desired domain of Nod2 and use the mutated polynucleotide in a targeting vector to study the role of such mutation in a transgenic organism. For example, peptides corresponding to one or more domains of Nod2, may be truncated or deleted and the corresponding Nod2 polynucleotide used in a targeting vector to develop a transgenic organism of the invention.

A Nod1 or 2 polynucleotide may be produced by recombinant DNA technology using techniques well known in the art. Such methods can be used to construct vectors containing a Nod2 polynucleotide. These methods include, for example, in vitro recombinant DNA techniques, synthetic techniques, and in vivo genetic recombination. See, for example, the techniques described in Sambrook et al., 1989, supra, and Ausubel et al., 1989.

A targeting vector of the invention comprises (a) a polynucleotide comprising SEQ ID NO:1; (b) a polynucleotide that hybridizes to the complement of a nucleic acid consisting of SEQ ID NO:1, under, for example, stringent conditions, e.g., hybridization to filter-bound DNA in 0.5 M NaHPO₄, 7% sodium dodecyl sulfate (SDS), 1 mM EDTA at 65° C., and washing in 0.1×SSC/0.1% SDS at 68° C. (Ausubel F. M. et al., eds., 1989, Current Protocols in Molecular Biology, Vol. 1, Green Publishing Associates, Inc., and John Willey & Sons, Inc., New York, at p. 2.10.3) (c) a polynucleotide that hybridizes to the complement of a nucleic acid consisting of SEQ ID NO:1, under less stringent conditions, such as moderately stringent conditions, e.g., washing in 0.2% SSC/0.2% SDS/0.1% SDS at 42° C. (Ausubel et al., 1989, supra); and (d) a fragment of any of (a) to (d) useful as primers and probes.

In addition to the polynucleotides identified herein (particularly as they relate to Nod1 and Nod2), homologs and orthologs of such Nod1 or 2 polypeptides and polynucleotides as may, for example, be present in other species, including humans, may be identified and used in the methods and compositions of the invention to obtain additional transgenic organisms.

The following examples are provided to further demonstrate the invention and do not limit the disclosure or the claims.

### EXAMPLES

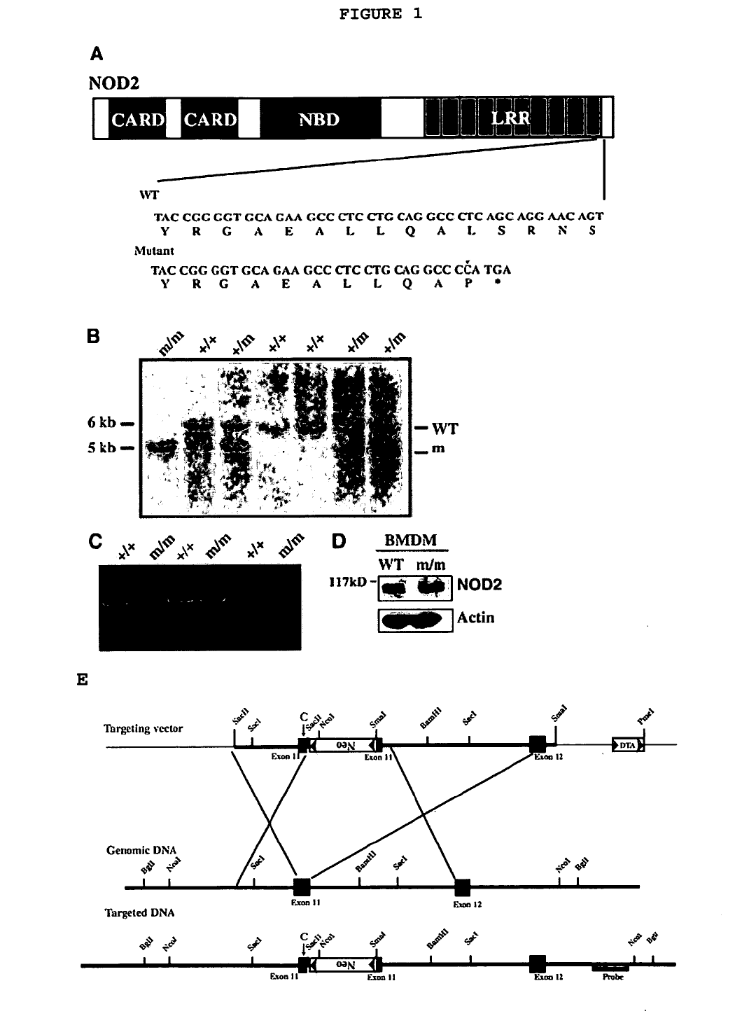

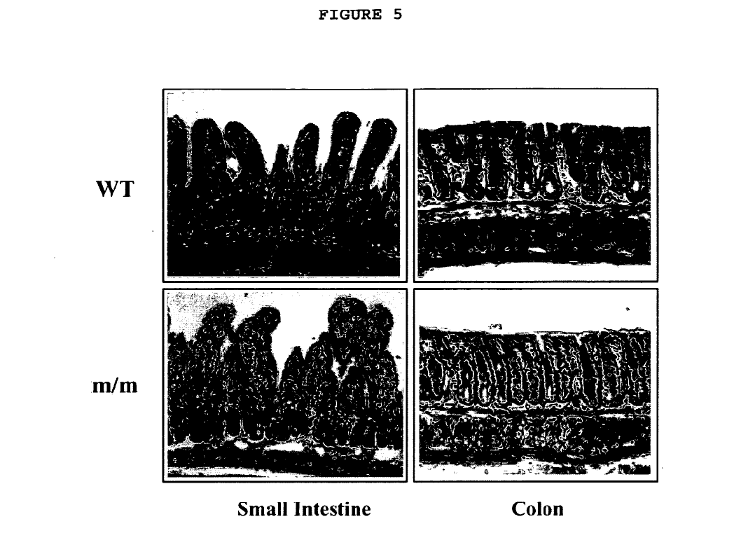

To address the problems associated with IBD (e.g., Crohn's disease(CD)) and illuminate the mechanism by which CD-associated Nod2 variants act, mice whose Nod2 locus harbors the homolog of the most common CD susceptibility allele, 3020insC, which encodes a truncated protein lacking the last 33 amino acids were generated. This was done through insertion of cytosine at position 2939 (corresponding to 3020 in human Nod2) of the Nod2 open reading frame (FIG. 1A, B). Homozygous Nod2^{2939iC} mice were obtained at the expected Mendelian ratio and did not show abnormalities of the gastrointestinal tract (FIG. 5), or other organs and were healthy. The mutation had no effect on Nod2 mRNA and protein amounts in bone-marrow derived macrophages (BMDM) (FIG. 1C, D).

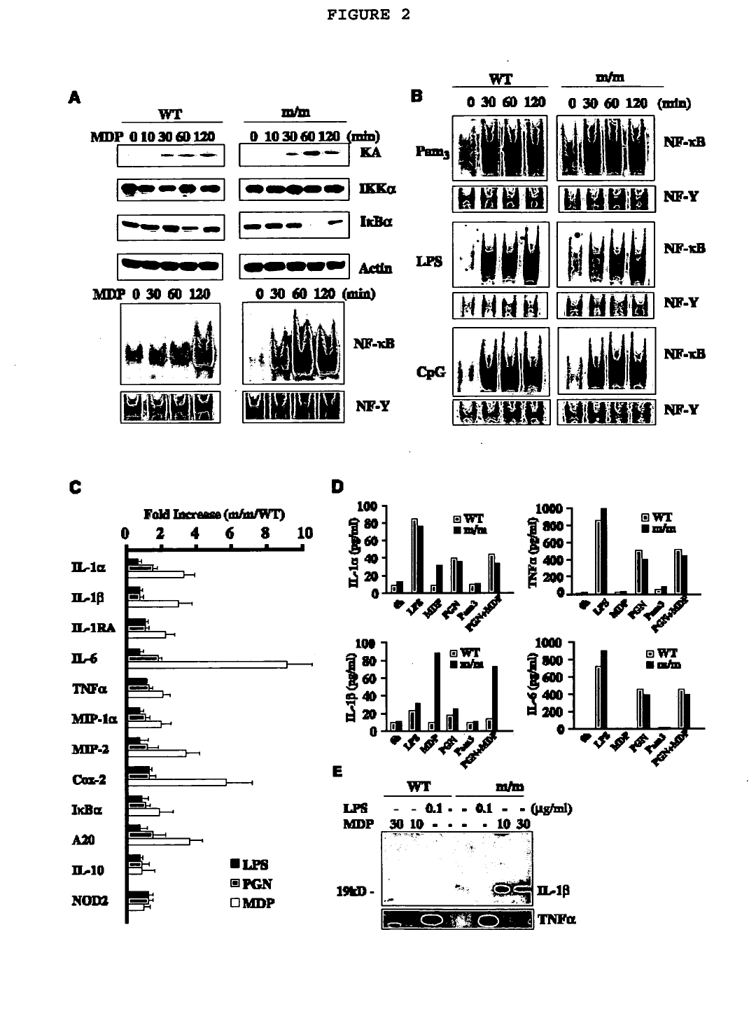

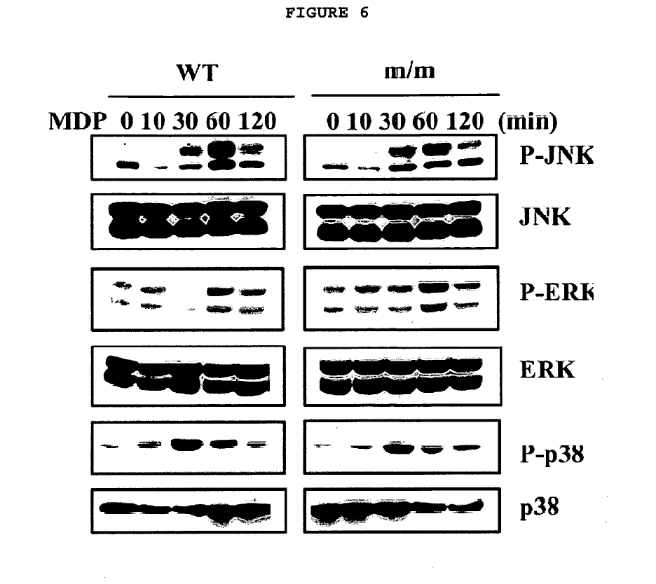

The effect of the Nod2^{2939iC} mutation on NF-κB activation in BMDM cultures was examined. IKK activity, IκBα degradation and NF-κB DNA binding activity were higher in MDP-stimulated Nod2^{2939iC} macrophages than in wild type (WT) cells (FIG. 2A). Only marginal differences in mitogen-activated protein kinases (MAPKs) were observed (FIG. 6). No genotype-specific differences in NF-κB activation were observed after macrophage treatment with other microbial components that activate Toll-like receptors (TLRs), including the TLR2-agonists Pam₃Cys and peptidoglycan (PGN), the TLR4-agonist lipopolysaccaride (LPS), and the TLR9-agonist non-methylated CpG-containing DNA (FIG. 2B). Expression of several NF-κB target genes was increased in MDP-treated Nod2^{2939iC} macrophages relative to WT counterparts (FIG. 2C). Only minor differences in expression of these genes were observed when macrophages were stimulated with LPS or PGN. Although MDP-induced gene expression of several cytokine genes was increased in Nod2^{2939iC} macrophages, only IL-1β secretion was significantly elevated in these cells relative to WT counterparts (FIGS. 2D, E, 7). Secretion of IL-1α was modestly elevated and neither IL-6 nor TNFα were secreted in response to MDP.

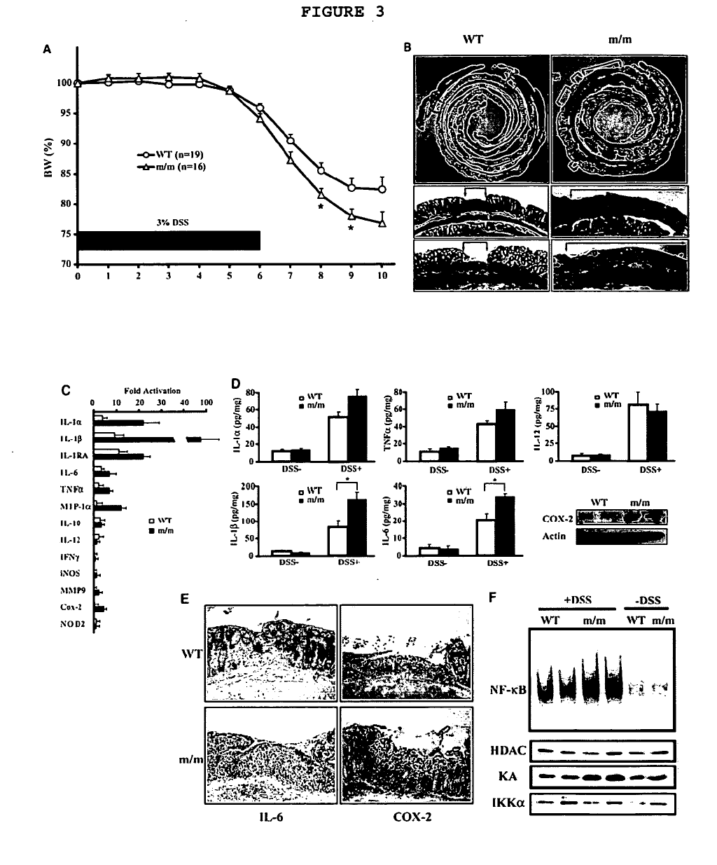

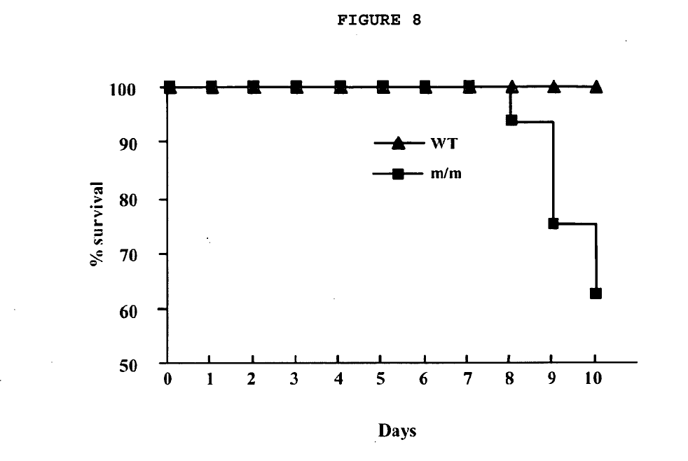

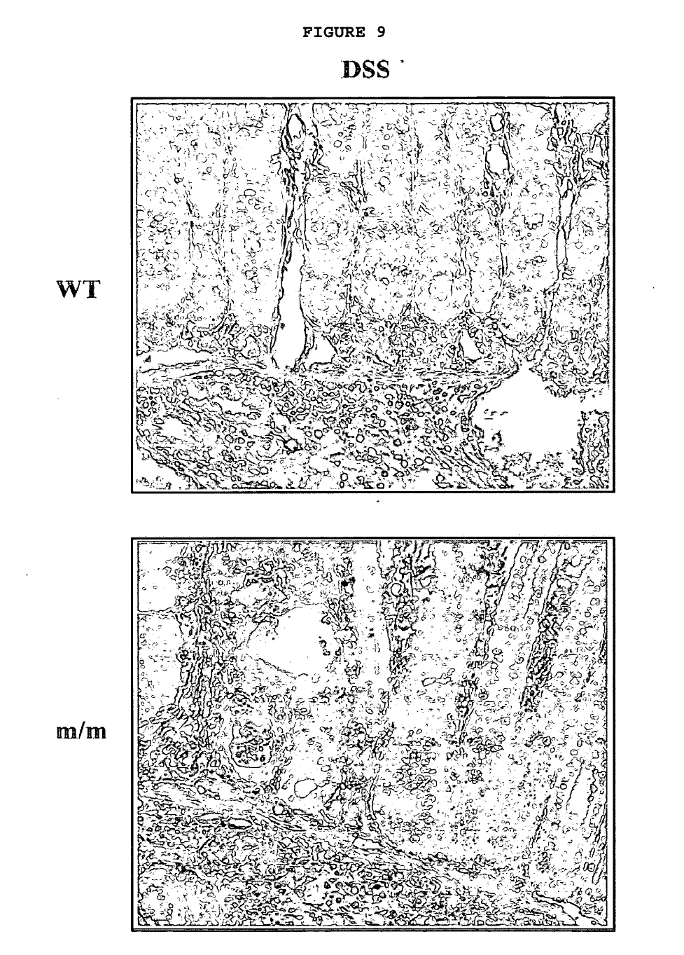

Macrophages involved in CD most likely reside in the lamina propria. To expose these cells to enteric bacteria, mice were treated with dextran sodium sulfate (DSS), an agent that kills mucosal epithelial cells and disrupts their barrier function, causing bacterial invasion. WT and homozygous Nod2^{2939iC} mice (8-12 weeks old) were given 3% DSS in drinking water for 6 days and monitored for weight loss, a characteristic of severe intestinal inflammation. After 8 days, body weight loss was greater in Nod2^{2939iC} mice relative to WT mice (FIG. 3A). Nod2^{2939iC} mice also exhibited increased mortality relative to WT mice (37.5% vs. 0%) (FIG. 8). Surviving mice of both genotypes regained body weight after day 11 and returned to normal 30 days after DSS administration. Histological analyses revealed that the severity and extent of inflammatory lesions in the colons of Nod2^{2939iC} mice were significantly (p<0.05) greater than in WT controls, with larger areas of ulceration and increased infiltration of F4/80-positive macrophages (FIG. 3B, FIG. 9).

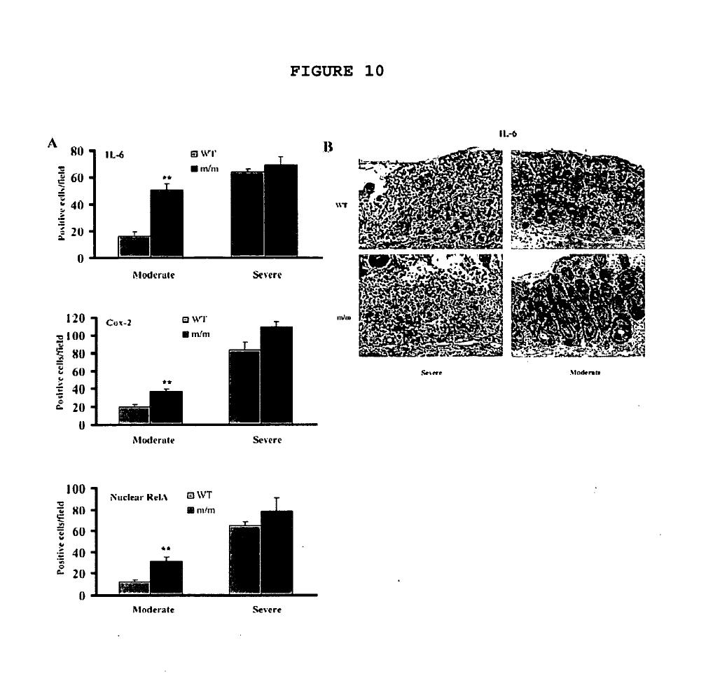

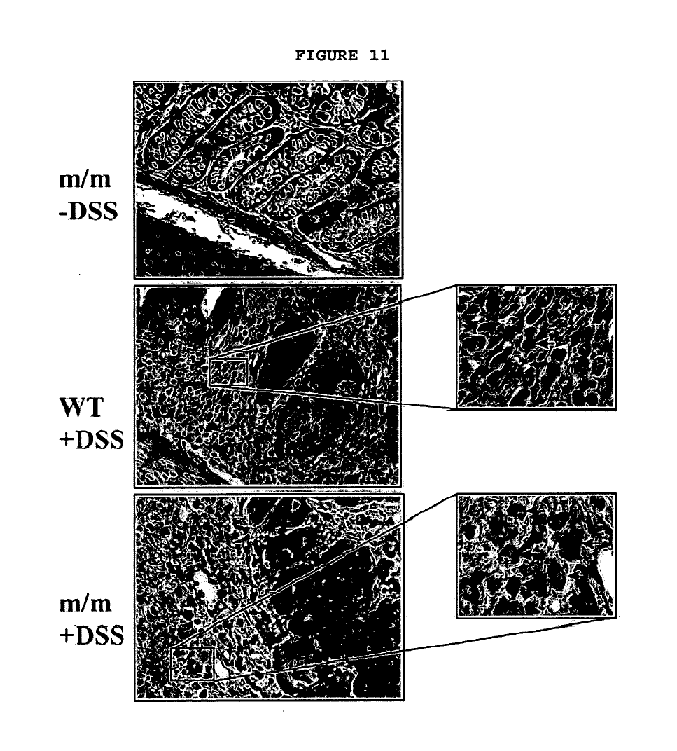

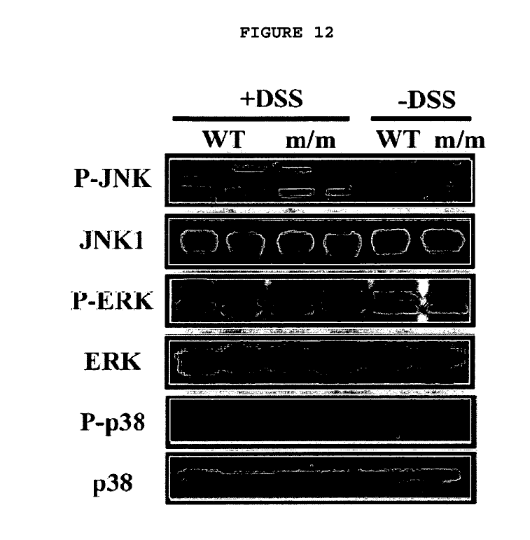

After DSS exposure, Nod2^{2939iC} homozygotes expressed greater amounts of mRNAs encoding pro-inflammatory cytokines and chemokines in their colons relative to WT mice (FIG. 3C). IL-1β, IL-6 and cyclooxygenase-2 (Cox-2) protein amounts were significantly higher in colons of DSS-treated Nod2^{2939iC} mice relative to WT counterparts (FIG. 3D). IL-6 and Cox-2 were predominantly expressed in F4/80-positive macrophages within inflammatory lesions (FIG. 3E, FIG. 10). IKK and NF-κB activities and RelA(p65) nuclear staining were also higher in colons of Nod2^{2939iC} mice than in the WT (FIG. 3F, FIG. 11). MAPK activation, however, was only marginally affected by the genotype (FIG. 12).

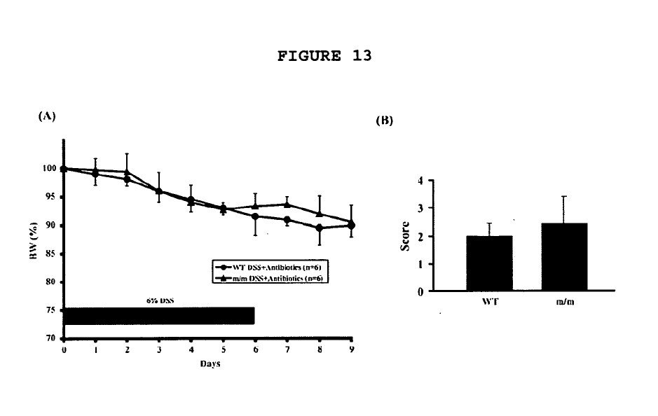

The intestinal inflammatory response to DSS is dramatically reduced by oral antibiotics, supporting involvement of enteric bacteria. When given a high dose of DSS (6%) without oral antibiotics, WT and Nod2^{2939iC} mice died within 9 days after DSS administration, but mice that received oral antibiotics survived and developed mild inflammation and weight loss, without any genotype-linked differences (FIG. 13). Thus, enteric bacteria elicit the inflammatory response to DSS and without such exposure, Nod2^{2939iC} mice do not behave differently from WT counterparts.

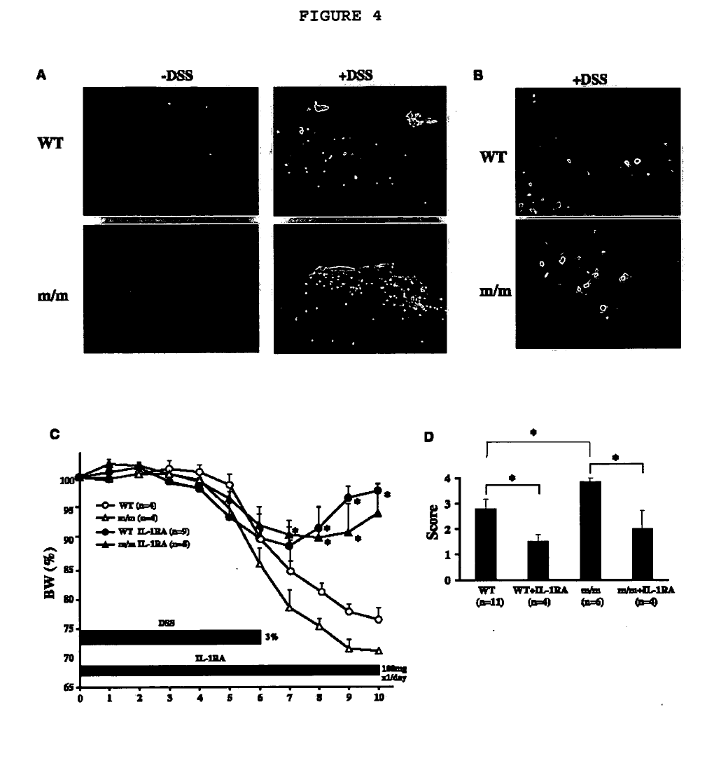

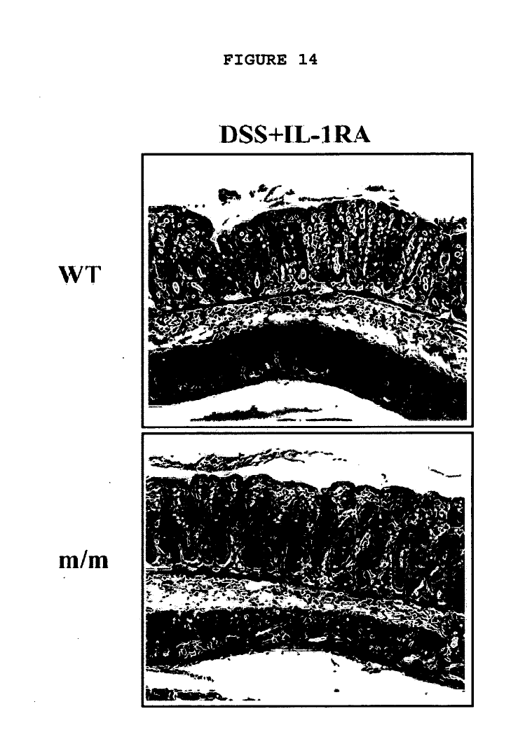

Exposure of macrophages to bacteria activates inflammatory and apoptotic caspases. More apoptotic cells, most of which positive for the F4/80 macrophage marker, were found in the lamina propria of DSS-treated Nod2^{2939iC} mice than in WT counterparts (FIG. 4A,B). Increased macrophage apoptosis is associated with activation of caspase-1, an enzyme required for secretion of mature IL-1β. Congruently, only background levels of secreted IL-1β were present in colons of untreated mice, but IL-1β concentrations were elevated after DSS treatment, particularly in Nod2^{2939iC} mice (FIG. 3D). Macrophage activation with LPS induces pro-IL-1β but its processing and release requires activation of caspase 1 by a different signal. LPS did not induce secretion of mature IL-1β in either Nod2^{2939iC} or WT macrophages, although it stimulated TNFα release (FIG. 2D, E). In contrast, MDP stimulated release of mature IL-1β, but not TNFα, by Nod2^{2939iC} macrophages. To determine whether IL-1β secretion may be involved in the increased inflammatory response to DSS in Nod2^{2939iC} mice, mice were injected once daily with IL-1 receptor antagonist (IL-1RA) from the start of DSS exposure. Average body weight loss and histological score were improved in IL-1RA treated mice and differences in weight loss (FIG. 4C) and inflammatory score (FIG. 4D, FIG. 14) between the genotypes were abolished.

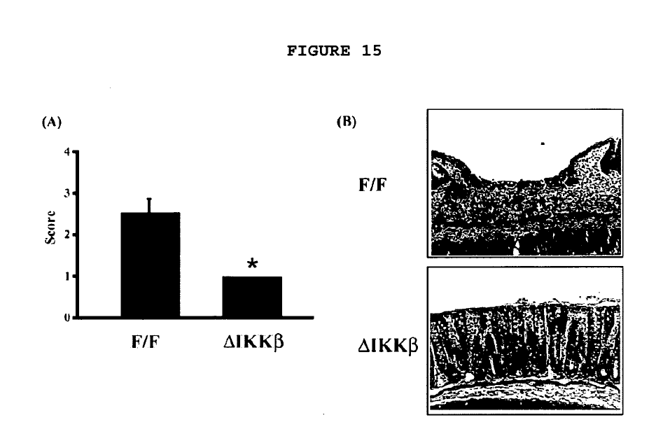

By contrast to the Nod2^{2939iC} mutation, deletion of Ikkβ in hematopoietic and myeloid cells reduced the inflammatory response to DSS (FIG. 15), but its deletion in enterocytes increased the inflammatory response to DSS.

Collectively, the results suggest that Nod2^{2939iC} is a gain-of-function allele, whose product induces elevated IKK and caspase-1 activation in response to MDP. Although NOD2 was suggested to be a negative regulator of TLR2, no effect of the Nod2^{2939iC} mutation on signaling by TLR2 was found as co-incubation of macrophages with MDP plus a TLR2 agonist (PGN) did not reduce to response to PGN (FIG. 2D). The inhibitory function hypothesis is also inconsistent with in vivo findings in Nod2 knockout mice, which did not show increased inflammation. The gain-of-function hypothesis is consistent with clinical observations made in CD patients.

The NF-κB signaling pathway induces many proinflammatory genes coding for cytokines and chemokines, including IL-1β, TNFα, and IL-6 and may therefore be an important pathogenic factor in CD. Although increased transcription of many NF-κB targets was observed, the results with IL-1β were unique as it was the only proinflammatory cytokine whose secretion in response to MDP was markedly elevated in Nod2^{2939iC} macrophages related to WT counterparts. The results suggest that IL-1β is indeed an important contributor to the increased colonic inflammation in Nod2^{2939iC} mice.

Although NF-κB was thought to be the major effector for Nod2, it should be noted that NF-κB is more effectively activated by bacterial products through TLRs (see FIG. 2). Thus NF-κB activation is not unique to Nod2 and its loss may not compromise NF-κB signaling in response to bacterial infection. Recently, TLR signaling and a certain amount of enteric bacteria were shown to be critical for maintenance of the intestinal barrier function, a function that was suggested to deteriorate in CD patients. However, maintenance of barrier function is unlikely to involve Nod2. By contrast, a unique function of Nod2, not provided by TLRs, is induction of IL-1β processing and release. This function can be mediated through the N-terminal CARD domains of Nod2, that can directly interact with caspase 1 or upstream caspases. Given the importance of IL-1β for the pathology of DSS-induced colitis in Nod2^{2939iC} mice, and the imbalance between IL-1β and IL-1RA in CD patients its role in CD pathogenesis is of importance.

Mice. An additional cytosine was inserted at position 2939 of the mouse Nod2 open reading frame via PCR. This insertion results in a frame-shift leading to premature termination and production of a truncated Nod2 protein as described for human NOD2^{3020iC}. A 1 kb fragment of Nod2 DNA containing a portion of the mutated exon 11 was inserted into the Sac1 site of a pBluescript targeting vector upstream of the neomycin resistance (Neo^{r}) gene, and a 3 kb fragment of Nod2 DNA containing the remainder of exon 11, the intron and exon 12 was inserted into a Sma1 site immediately downstream. The targeting vector also contained a diphtheria toxin (DTA) gene for negative selection. The DTA gene contains the Pme1 site that was used to linearize the vector. Linearized vector DNA was electroporated into ES cells. Approximately 200 G418-resistant clones were collected and screened by PCR to identify homologous integrants at the Nod2 locus. Several positive clones were identified, and one of them was injected into C57BL/6 blastocysts. Male chimeras crossed with C57BL/6 females gave rise to heterozygous Nod2^{+/2939iC} mice that were intercrossed to obtain homozygous Nod2^{2939iC} mutants. Genotypes were analyzed by PCR and confirmed by Southern Blot analysis of Nco1-digested tail genomic DNA (10 μg), yielding 5.5 and 4.2 kb fragments for the Nod2+ and Nod2^{2939iC} alleles respectively.

DSS colitis, IL-1RA treatment and histological scoring. Mice (8-12 weeks old) were given DSS (ICN Biomedicals Inc.) in the drinking water for 6 days as indicated and placed on regular water thereafter. When indicated, mice were also treated with neomycin sulfate (1.5 g/L) and metronidazole (1.5 g/L) (both from Sigma) in the drinking water or injected i.p. with either IL-1RA (Kineret®, Amgen Inc.) (100 mg/kg) in PBS or PBS alone once daily throughout the experiment. For histological and gene expression analyses, mice were sacrificed either before or 11 days after initiation of DSS treatment. Otherwise, mice were observed for 30 days after initiation of treatment. Histological scoring of fixed (10% formaldehyde) and sectioned (paraffin embedded) tissue was performed in a blinded manner. The scores were: 0=normal, 1=moderate mucosal inflammation without ulcer, 2=severe mucosal inflammation with ulcer (<1 mm) or no ulcer, 3=severe mucosal inflammation with ulcer (1-3 mm), 4=severe mucosal inflammation with ulcer (>3 mm).

Macrophage culture and treatment. BMDMs were cultured as described (Park et al., Science 297: 2048, 2002). Confluent cultures were treated with different bacterial components including MDP (Bachem), synthetic peptidoglycan-Pam₃Cys (InvivoGen), natural gram positive peptidoglycan (from S. aureus, Sigma), LPS (from E. coli, Sigma), and CpG-DNA (TIB MOLBIOL). At the indicated time points the cells or culture supernatants were collected and used to prepare cytoplasmic and nuclear protein extracts or total cellular RNA.

IKK and NF-κB assays. IKK activity was determined by an immunecomplex kinase assay using an anti-IKKγ antibody (PharMingen) for immunoprecipitation and anti-IKKα antibody (Upstate Biologicals) to monitor recovery. NF-κB DNA binding activity was determined by electrophoretic mobility shift assay.

Gene expression analyses. Protein lysates were prepared from tissues and cultured macrophages, separated by SDS-polyacrylamide gel electrophoresis, transferred to Immobilon membranes (Millipore) and analyzed by immunoblotting. Total cellular RNA was extracted using TRIZOL (Invitrogen). cDNA was generated using SuperScript II (Invitrogen) and the amounts of the different mRNAs were measured by real-time PCR using GAPDH mRNA for normalization. Primer sequences are available upon request. Cytokine levels were measured using enzyme linked immunoadsorbent assays (ELISA).

Immunohistochemistry. Colons were fixed in 10% formaldehyde, dehydrated, embedded in paraffin and sectioned (5 μm). Sections were deparaffinized, rehydrated, and treated with 3% H₂O₂ in phosphate-buffered saline (PBS) and incubated overnight at 4° C. with anti-IL-6 (R&D systems), anti-Cox-2 (Cayman Chemical), anti-F4/80 (Caltag) antibodies or identical concentrations of isotype matched control antibodies. Binding of primary antibody was detected with biotin-labeled anti-rabbit IgG or anti-rat IgG antibodies (1:500 dilution; Vector Laboratories), followed by streptavidin-horseradish peroxidase reaction and visualization with 3,3′-diaminobenzidine (Sigma) and counterstaining with hematoxylin. TUNEL staining was performed.

Statistical Analysis. Differences between means were compared by Student t tests. p values <0.05 were considered significant.

A number of embodiments of the invention have been described. Nevertheless, it will be understood that various modifications may be made without departing from the spirit and scope of the invention. Accordingly, other embodiments are within the scope of the following claims.

What is claimed is:

1. A method of inducing inflammatory bowel disease (IBD)-like symptoms in an animal, comprising contacting a transgenic non-human animal comprising a mutant Nod gene product with an agent that induces IBD-like symptoms.

2. The method of claim 1, wherein the IBD-like symptoms comprise Crohn's disease symptoms.

3. The method of claim 1, wherein the symptoms comprise elevated interleukin-1β and/or NF-κB activation compared to control animals.

4. The method of claim 1, wherein the agent comprises muramyl dipeptide (MDP) and/or dextran sodium sulfate (DSS).

5. The method of claim 1, wherein the mutant Nod gene product is a mutant Nod2 gene product.

6. The method of claim 5, wherein the mutant Nod2 gene product lacks the C-terminal region of the wild-type Nod2 gene product.

7. The method of claim 6, wherein the mutant Nod2 gene product lacks the C-terminal 33 amino acids of the wild-type Nod2 gene product.

8. The method of claim 1, wherein the transgenic non-human animal is a mouse.

9. The method of claim 8, wherein the mutant Nod gene product comprises a Nod2 polynucleotide having an insertion of cytosine at position 2939.

10. The method of claim 1, wherein the transgenic non-human animal is a transgenic Nod2^{2939iC} mouse.

11. A method of generating an inflammatory bowel disease animal model, comprising:
(i) providing an embryonic stem (ES) cell from a relevant animal species comprising a Nod2 gene;
(ii) providing a targeting vector comprising a polynucleotide having a mutant Nod2 polynucleotide capable of homologous recombination with the Nod2 gene;
(iii) introducing the targeting vector into the ES cells under conditions where the Nod2 gene undergoes homologous recombination with the targeting vector to produce a mutant Nod2 gene;
(iv) introducing the ES cells carrying a mutant Nod2 gene into a blastocyst;
(v) implanting the blastocyst into the uterus of pseudopregnant female;
(vi) delivering animals from said female; and
(vii) selecting for transgenic Nod2 mutant animals.

12. The method of claim 11, wherein the animal is a mouse.

13. The method of claim 11, wherein the Nod2 mutant animal comprise elevated interleukin-1β and/or NF-κB activation compared to wild-type animals in the presence of MDP.

14. The method of claim 11, wherein the mutant Nod2 polynucleotide encodes a polypeptide product lacking the C-terminal region of the wild-type Nod2 gene product.

15. The method of claim 14, wherein the mutant Nod2 polynucleotide encodes a polypeptide product lacking the C-terminal 33 amino acids of the wild-type Nod2 gene product.

16. The method of claim 12, wherein the mutant Nod2 polynucleotide comprises an insertion of cytosine at position 2939 of SEQ ID NO:2.

17. The method of claim 16, wherein the transgenic Nod2 mutant animal is a transgenic Nod2^{2939iC} mouse.

18. A transgenic non-human animal produced by the method of claim 11.

19. A transgenic non-human animal comprising a mutant Nod gene, wherein the transgenic non-human animal demonstrates a phenotype, when contacted with MDP, of increased activation of NF-κB and/or increased interleukin-1β secretion.

20. The transgenic non-human animal of claim 19, wherein the Nod gene is a Nod2 gene.

21. The transgenic non-human animal of claim 20, wherein the mutant Nod2 gene encodes for a polypeptide that lacks a C-terminal portion of the wild-type Nod2 polypeptide.

22. The transgenic non-human animal of claim 21, wherein the mutant Nod2 gene encodes a polypeptide that lacks the C-terminal 33 amino acids of the wild-type Nod2 polypeptide.

23. The transgenic non-human animal of claim 19, wherein the animal is a mouse.

24. The transgenic non-human animal of claim 23, wherein the mouse is a Nod2^{2939iC} transgenic mouse.

25. A cell line derived from a transgenic non-human animal of claim 19.

26. A cell of claim 25, wherein the cell is selected from the group consisting of stem cells, intestinal epithelial cells and bone marrow derived cells.

27. A method of screening an agent for its efficacy in ameliorating the symptoms of inflammatory bowel disease (IBD), comprising
administering a candidate agent to a non-human transgenic animal comprising a mutated Nod gene product, wherein the non-human transgenic animal is characterized by having elevated interleukin-1β levels when contacted with MDP; and
comparing the symptoms of IBD in the non-human transgenic animal to one or more control animals, wherein a decrease in symptoms of IBD in the animal treated with the test agent indicates efficacy of the agent.

28. The method of claim 27, wherein the IBD comprises symptoms of Crohn's disease.

29. The method of claim 27, wherein the non-human transgenic animal comprises a mutation in Nod2, wherein the mutation results in an early termination and/or C-terminal truncation of the Nod2 polypeptide.

30. The method of claim 27, wherein the test agent is selected from the group consisting of small molecules, peptides, polypeptides, proteins, peptidomimetics, antibodies, nucleic acids, antisense nucleic acids, and ribozymes.

31. The method of claim 27, wherein the agent is an antibody that interacts with a CARD domain of a Nod polypeptide.

32. A method of inhibiting an inflammatory bowel disease (IBD) in a subject having or at risk of having such a disease comprising:
contacting the subject with an agent that inhibits the activity of an N-terminal CARD domain of a Nod polypeptide.

33. The method of claim 32, wherein the agent inhibits the interaction of the N-terminal CARD domain with its ligand.

34. The method of claim 32, wherein the agent inhibits the interaction of the N-terminal CARD domain with a caspase.

35. The method of claim 34, wherein the caspase is caspase-1.

36. The method of claim 32, wherein the agent inhibits the interaction of the N-terminal Card domain with RIP2.

37. The method of claim 32, wherein the agent is an antibody that binds to a member selected from the group consisting of the CARD domain of a Nod polypeptide, caspase-1, and RIP2.

38. The method of claim 32, wherein the IBD is Crohn's disease.

39. The method of claim 32, wherein the IBD is Blau syndrome.

40. The method of claim 32, wherein the Nod is Nod1.

41. The method of claim 32, wherein the Nod is Nod2.


SEQUENCE LISTING (3 sequences)

SEQ ID NO: 1 - DNA, 36 bp, Artificial Sequence
  Polynucleotide Fragment
taccggggtgcagaagccctcctgcaggccccatga

SEQ ID NO: 2 - DNA, 3042 bp, Mus musculus
  CDS (1)..(3042)
atgtgctcacaggaagagttccaggcacagaggagccagctggtggcactgctgatctcagggtccttggagggctttgagagcatcttagactggctgctgtcttgggatgtgctctccagggaggactatgagggcctcagcctccctggccaacctctctcccattctgccaggcgtctgctggacacagtctggaacaagggtgtttggggctgtcagaagctcctcgaagctgtgcaggaggcacaggccaacagtcatacctttgaactgtatgggtcctgggacactcactcccttcatccaaccagagacctgcagagtcaccggccagccattgtgaggagactctacaaccatgtagaagccatgctggagctggcaagggagggggggtttctgagccagtacgagtgtgaggagatcaggctgccaatcttcacgtcgtctcagagggcaagaaggctgcttgatctcgctgcggtgaaggccaatggactggctgccttccttctacagcacgtcagggaactgccagctccactgcctctgccttacgaggctgctgagtgtcagaagttcatatctaagctgaggaccatggtgttggctcagtctcgcttcctcagtacttacgatgggtcggagaatctttgcctggaggatatatacacggagaacatcttggagctgcggactgaagtgggcacagccggggccttgcagaagagccctgccatcctgggcctggaggacctctttgatacccatggtcacctgaacagagatgccgacaccatactggtggtgggcgaagcaggcagtggcaagagcactcttctgcagcgtttgcacctgctgtgggcaacagggaggagcttccaggagtttctcttcattttcccattcagctgccgacagttgcaatgcgtggccaaaccgctgtccctgaggacgctgctctttgagcactgctgctggcctgatgtcgctcaggacgatgtcttccagttccttcttgaccatcctgaccgtgtcctgttaacctttgatggcttggacgagttcaagttccggttcaccgaccgggagcgccactgctctccaattgaccccacgtcagtccagactctgctcttcaaccttctccaggggaacctgctgaagaatgcctgcaaggtgctgaccagccgtccggatgctgtgtcagcgctcctcaggaagttcgtccgtacagagtgccaactgaagggcttctctgaagagggcatccaactgtacctgagaaagcaccaccgggaacctggggtggcagaccgcctcatccagctgatccaagccacctcagccctgcatggtttgtgccacctccctgtcttctcttggatggtgtccagatgccaccgggaacttctgctgcagaacaggggcttcccaacaaccagcacggacatgtacctcctgatcctgcagcatttcctgctgcatgcctcccctccggactcctccccactcggtctgggacctggactcctccaaagccggctctccaccctcctgcaccttggccacctggctctccggggcctggccatgagctgctatgtgttctcagcccagcagctccaggcagctcaggttgactctgatgatatttctcttggtttcctggtacgtgcccaaagtagtgtgcccgggagcaaggcgcccctggagttcctgcacattaccttccagtgcttttttgccgctttctacttggctgtcagtgctgacacatcagtggcctctctcaagcaccttttcagctgtggccggctgggcagctcactgctgggaaggctgctgcccaacctgtgtatccagggctccagagtcaagaagggcagcgaagcagccctgctgcagaaggctgagccacacaacctgcagatcacagcagccttcctagcaggtctgttgtcccagcagcatcgggacctgttggctgcatgccagatctccgaaagggtgctgctccagcgtcaggcacgtgcccgctcatgtctggcccacagcctccgcgagcacttccattccatcccgcctgccgttcccggtgagaccaagagcatgcatgctatgccgggctttatttggctcatccggagcctgtacgagatgcaggaggagcagttggcccaggaggctgtccgtcgcttggacatcgggcacctgaagttgacattttgcagagtgggccctgcagagtgtgctgcgctggcctttgtactgcaacatctccagcggcctgtggccctacagctggattacaactctgtgggagatgtcggagtggaacagctgcgaccgtgccttggggtctgcacagctctgtatttgcgagataacaatatctcagaccgaggtgcccgcacgctggttgagtgtgctcttcgctgtgagcagctgcagaaactagctctcttcaacaacaaactcacggatgcgtgcgcctgctccatggccaagctccttgcacacaagcagaacttcttgtccctgagggtggggaacaatcacatcacagccgctggagccgaggtgctggcccagggactcaagagcaacacctccctgaagttcctggggttctggggcaacagcgtgggtgataagggcacccaagccctggctgaagttgtagccgaccaccagaacctaaagtggctcagcttggtaggaaacaacattggcagcatgggtgcccaagccctagcactgatgctggagaagaacaagtcactagaggagctctgcctagaggaaaaccatatctgtgacgaaggggtatattctctcgcagaaggactcaagagaaattcaactttgaaattcttgaaactgtccaacaatggcatcacctaccggggtgcagaagccctcctgcaggccctcagcaggaacagtgccattctggaggtttggcttcgagggaacacattctctttggaggaaatccaaacactgagctccagggacgccagactcttgttgtga

SEQ ID NO: 3 - protein, 1013 aa, Mus musculus
MCSQEEFQAQRSQLVALLISGSLEGFESILDWLLSWDVLSREDYEGLSLPGQPLSHSARRLLDTVWNKGVWGCQKLLEAVQEAQANSHTFELYGSWDTHSLHPTRDLQSHRPAIVRRLYNHVEAMLELAREGGFLSQYECEEIRLPIFTSSQRARRLLDLAAVKANGLAAFLLQHVRELPAPLPLPYEAAECQKFISKLRTMVLAQSRFLSTYDGSENLCLEDIYTENILELRTEVGTAGALQKSPAILGLEDLFDTHGHLNRDADTILVVGEAGSGKSTLLQRLHLLWATGRSFQEFLFIFPFSCRQLQCVAKPLSLRTLLFEHCCWPDVAQDDVFQFLLDHPDRVLLTFDGLDEFKFRFTDRERHCSPIDPTSVQTLLFNLLQGNLLKNACKVLTSRPDAVSALLRKFVRTECQLKGFSEEGIQLYLRKHHREPGVADRLIQLIQATSALHGLCHLPVFSWMVSRCHRELLLQNRGFPTTSTDMYLLILQHFLLHASPPDSSPLGLGPGLLQSRLSTLLHLGHLALRGLAMSCYVFSAQQLQAAQVDSDDISLGFLVRAQSSVPGSKAPLEFLHITFQCFFAAFYLAVSADTSVASLKHLFSCGRLGSSLLGRLLPNLCIQGSRVKKGSEAALLQKAEPHNLQITAAFLAGLLSQQHRDLLAACQISERVLLQRQARARSCLAHSLREHFHSIPPAVPGETKSMHAMPGFIWLIRSLYEMQEEQLAQEAVRRLDIGHLKLTFCRVGPAECAALAFVLQHLQRPVALQLDYNSVGDVGVEQLRPCLGVCTALYLRDNNISDRGARTLVECALRCEQLQKLALFNNKLTDACACSMAKLLAHKQNFLSLRVGNNHITAAGAEVLAQGLKSNTSLKFLGFWGNSVGDKGTQALAEVVADHQNLKWLSLVGNNIGSMGAQALALMLEKNKSLEELCLEENHICDEGVYSLAEGLKRNSTLKFLKLSNNGITYRGAEALLQALSRNSAILEVWLRGNTFSLEEIQTLSSRDARLLL